(19) United States
(12) Patent Application Publication
(10) Pub. No.: US 2025/0325728 A1
(43) Pub. Date: Oct. 23, 2025

(54) Oxidatively Degrading and ROS Scavenging Polymers

(71) Applicant: BRIDGEWAY INNOVATIONS, LLC, College Station, TX (US)

(72) Inventors: Landon D. Nash, Park City, UT (US); Tyler J. Touchet, Cypress, TX (US); Duncan J. Maitland, College Station, TX (US); Balakrishna Haridas, College Station, TX (US)

(21) Appl. No.: 19/024,055

(22) Filed: Jan. 16, 2025

Related U.S. Application Data
(63) Continuation-in-part of application No. PCT/US2023/070302, filed on Jul. 17, 2023.
(60) Provisional application No. 63/389,964, filed on Jul. 18, 2022.
(60) Provisional application No. 63/497,739, filed on Apr. 23, 2023.

(51) Int. Cl. A61L 27/18; A61L 27/56; C08G 18/52

(52) U.S. Cl. CPC A61L 27/18; A61L 27/56; C08G 18/52; A61L 2430/02

(57) ABSTRACT

The proposed invention is a reactive oxygen species scavenging polymer system with biodegradable and biodurable embodiments for biomedical applications. Several embodiments are tailored for patients with high oxidative stress due to comorbidities (diabetes, obesity, peripheral neuropathy, osteoporosis, osteopenia, chronic kidney disease, neurodegenerative diseases, and cardiovascular disease). Other embodiments function independent of atypical oxidative stress to provide a biodegradable biomaterial system. Several embodiments include thiourethane synthetic schemes to create biomaterials for applications requiring increased toughness.

### CROSS-REFERENCE TO RELATED APPLICATION

This application is a continuation-in-part of international application PCT/US2023/070302, filed on Jul. 17, 2023 which claims priority to both: (a) U.S. Provisional Patent Application No. 63/389,964, filed on Jul. 18, 2022, and entitled “Oxidatively Degrading Thermoplastic Polyurethanes and 3D Printable Degrading Polyurethane Resins”, and (b) U.S. Provisional Patent Application No. 63/497,739, filed on Apr. 23, 2023, and entitled “Oxidatively Degrading and ROS Scavenging Polymers for Medical Applications”. The content of each of the above applications is hereby incorporated by reference.

### BACKGROUND

Injection molding, extrusion, 3D printing, electrospinning, solvent casting, film blowing, machining, chemical foam blowing, supercritical gas foaming, porogen templating; diabetic ulcer patches, long time-scale degrading ligament fixation, biodegradable vascular stents, surgical sutures, orthopedic anchors, surgical meshes, biodegradable elastomer, implant coatings, drug delivery vehicle, bone graft, hydrocephalus shunt, glaucoma shunt, osteoporosis/osteopenia femoral neck stabilizer, soft tissue staple, orthopedic staple, woven tissue interface, tissue adjunct, nerve sheath, spinal disk replacement, vertebral fusion, would dressings, cartilage regeneration.

### BRIEF DESCRIPTION OF THE DRAWINGS

Features and advantages of embodiments of the present invention will become apparent from the appended claims, the following detailed description of one or more example embodiments, and the corresponding figures. Where considered appropriate, reference labels have been repeated among the figures to indicate corresponding or analogous elements.

FIG. 1 depicts an embodiment of a tertiary amine polyurethane synthesis.

FIG. 2 depicts an embodiment of a tertiary amine polyurea synthesis.

FIGS. 3A, 3B, 3C depict embodiments of degradable repeat unit oxidation pathways.

FIG. 4 depicts an embodiment of a (a) crosslinkable, oxidatively degradable tertiary amine containing thermoplastic, (b) radical formation due to ionizing radiation exposure, and (c) direct crosslinking of thermoplastic pendant groups.

FIG. 5 depicts an embodiment of (a) a crosslinkable, oxidatively degradable tertiary amine containing thermoplastic, (b) radical formation due to ionizing radiation exposure via radiation sensitizing agents, and (c) crosslinking.

FIG. 6 depicts embodiments of formation of therapeutic agent macromers. The top formation shows a radiation sensitizing crosslinker formed from estradiol. The bottom formation shows a thiol-ene crosslinking agent formed from estradiol.

FIG. 7 depicts embodiments of additional therapeutic agent macromers for incorporating antimicrobial p-coumaric acid into a polymer network. Either as a crosslinking agent between pendant thermoplastic alkene groups, or via direct polymerization.

FIG. 8 depicts an embodiment of covalent crosslinking of polyurethane thermoplastic chains using a thiol terminated estradiol macromer.

FIG. 9 depicts an embodiment of thermoset synthesis using a linear tertiary amine macromer with alkene termination combined with a polythiol crosslinker.

FIG. 10 depicts an embodiment of branched tertiary amine macromers with alkene termination reacted with dithiol or polythiol monomers for thermoset polymer fabrication.

FIG. 11 depicts embodiments of interchangeable monomer schemes to produce alkene terminated macromers with urethane, urea, aliphatic, and aromatic linkages.

FIG. 12 depicts embodiments of interchangeable monomer schemes to produce alkene terminated macromers with thiourethane, urethane, and tertiary amine linkages.

FIG. 13 depicts an embodiment of synthesis of sulfide macromers for increased ROS sequestering when subsequently polymerized into a polymer system.

FIG. 14 depicts an embodiment of a secondary amine containing alkene terminated macromer synthesis.

The upper portion of FIG. 15 depicts embodiments of configurations of biomaterials with single and double biodegradation mechanisms. The lower portion of FIG. 15 shows comparative hydrolytic rates based on the degree of material oxidation.

FIG. 16 depicts an embodiment of a population of hydrolytically labile macromers with differing rates of hydrolytic release of a therapeutic agent.

FIG. 17 depicts an embodiment of isocyanate-free synthesis of a tertiary amine containing an oxidatively labile thermoplastic polymer and an example of subsequent functionalization with an antimicrobial agent via carboxylic acid esterification.

FIG. 18 depicts an embodiment of a thiourethane reaction.

FIG. 19 depicts an embodiment of synthesis of thiourethane macromers from thioglycerol and HDI.

FIG. 20 depicts an embodiment of synthesis of an oxidatively labile, tertiary amine containing thermoset using a thiourethane macromer, tertiary amine containing diol, and an aliphatic diisocyanate.

FIG. 21 depicts an embodiment of a DMA curve of a thermoset polythiourethane polymer.

FIG. 22 depicts an embodiment of synthesis of a hydrolytically responsive linear aliphatic polythiourethane repeat unit derived from diisocyanate and dithiol monomers catalyzed with a tertiary amine group.

FIG. 23 depicts an embodiment of polymer networks with short degradable repeat unit thermoplastics, medium degradable thermoplastic repeat units from copolymers, or long degradable thermoplastic repeat units.

FIGS. 24A, 24B, 24C, 24D depict embodiments of polymer architectures that include thermoplastic chains that have been subsequently crosslinked or lightly crosslinked thermosets to form elastomeric materials.

FIGS. 25A, 25B, 25C depict embodiments that include highly crosslinked thermosets.

FIG. 26A is an embodiment of a HIPE (emulsion) macromer system. FIG. 26B depicts an example of the scheme of FIG. 26A.

FIG. 27 depicts embodiments of alkene terminated, tertiary amine containing macromers useful for thermoset and thermoplastic systems.

FIG. 28A depicts an embodiment of a monomer scheme to synthesize a thermoplastic thioether with tertiary amine functional groups for oxidative sequestration and oxidative degradation in-vivo. FIG. 28B depicts an example of the scheme of FIG. 28A.

### DETAILED DESCRIPTION

Reference will now be made to the drawings wherein like structures may be provided with like suffix reference designations. In order to show the structures of various embodiments more clearly, the drawings included herein are diagrammatic representations of structures. Thus, the actual appearance of the fabricated structures, for example in a photo, may appear different while still incorporating the claimed structures of the illustrated embodiments (e.g., walls may not be exactly orthogonal to one another in actual fabricated devices). Moreover, the drawings may only show the structures useful to understand the illustrated embodiments. Additional structures known in the art may not have been included to maintain the clarity of the drawings. For example, not every layer of a device is necessarily shown. “An embodiment”, “various embodiments” and the like indicate embodiment(s) so described may include particular features, structures, or characteristics, but not every embodiment necessarily includes the particular features, structures, or characteristics. Some embodiments may have some, all, or none of the features described for other embodiments. “First”, “second”, “third” and the like describe a common object and indicate different instances of like objects are being referred to. Such adjectives do not imply objects so described must be in a given sequence, either temporally, spatially, in ranking, or in any other manner. “Connected” may indicate elements are in direct physical or electrical contact with each other and “coupled” may indicate elements co-operate or interact with each other, but they may or may not be in direct physical or electrical contact. Phrases such as “comprising at least one of A or B” include situations with A, B, or A and B

U.S. Ser. No. 11/530,291 relates to chemical polymer compositions, methods of synthesis, and fabrication methods for devices regarding polymers capable of displaying shape memory behavior (SMPs) and which can first be polymerized to a linear or branched polymeric structure, having thermoplastic properties, subsequently processed into a device through processes typical of polymer melts, solutions, and dispersions and then crossed linked to a shape memory thermoset polymer retaining the processed shape.

U.S. Ser. No. 11/613,603 provides an embodiment that includes a platform shape memory polymer system. Such an embodiment exhibits a blend of tunable, high performance mechanical attributes in combination with advanced processing capabilities and good biocompatibility.

Previously described systems contemplate a linear thermoplastic polyurethane polymer with unsaturated carbon-carbon motifs (either pendant groups or in the backbone) that can be crosslinked at the alkene centers with ionizing radiation, or between alkene groups using a thiol crosslinking agent and thiol-ene click chemistry. This material system enables thermoset systems that are superior for shape memory properties while also having the processing capabilities of traditional thermoplastics. A possible limitation with this system is a lack of ROS sequestration or biodegradation in the polymer backbone. Some embodiments may imply hydrolytic degradation and ROS sequestering at a thiol-ene generated crosslinking site, but these labile crosslink sites leave a high molecular weight thermoplastic implant after crosslink scission, possibly limiting this system's use in medical applications requiring full material degradation and clearance, such as tissue engineering devices.

Previously described systems also contemplate a linear thermoplastic polyurethane polymer containing unsaturated carbon-carbon motifs, with an emphasis on alkene pendant groups, that can be crosslinked at the alkene groups using a thiol crosslinking agent and thiol-ene click chemistry. This material system enables thermoset systems that are superior for shape memory properties while also having the processing capabilities of traditional thermoplastics. A possible limitation with this system is a lack of ROS sequestration or biodegradation in the polymer backbone. Embodiments imply hydrolytic degradation and ROS sequestering at a thiol-ene generated crosslinking site, but these labile crosslink sites may still leave a high molecular weight thermoplastic implant after crosslink scission, limiting this system's use in medical applications requiring full material degradation and clearance, such as tissue engineering devices.

Previously described systems contemplate incorporating phenolic acids into a thermoset polyurethane system to impart antimicrobial properties. Embodiments include direct phenolic acid incorporation into the network by reacting with isocyanates, or functionalization of polyols with monofunctional phenolic acids via esterification prior to the polyurethane synthesis. This material system is possibly only applicable to thermoset polymers, limiting the manufacturing flexibility for medical device fabrication. There are possibly no proposed embodiments for thermoplastic material systems that are also oxidatively biodegradable. There are also no embodiments for pendant functionalization of polyfunctional phenolic acids.

Previously described systems contemplate thermoset polyurethanes manufactured from tertiary amine polyols (functionality>2) to create networked polymer structures. The high degree of covalent crosslinking enables superior shape memory properties. Disclosed embodiments may include polymers that are intentionally biostable due to the conscious exclusion of ester and ether groups. These polymers may be intentionally high crosslinked and inherently exclude thermoplastic embodiments.

Methyl diethanolamine (MDEA) has been used in polyurethane systems to form ionomers in water dispersions for coating applications. These ionomers are also used to alter mechanical properties. The authors are not aware of a polyurethane system derived from MDEA that contemplates gravimetric mass loss and biodegradation when exposed to an oxidative environment (in-vitro or in-vivo).

Embodiments include a material system platform that enables independent incorporation of multiple functional properties for biomedical applications including: biodegradation (hydrolytic or oxidative pathways) within the polymer backbone and/or crosslinking segments, selective biodurability (within the backbone or crosslinking segments), thermoplastic or thermoset polymer network configurations, selective reactive oxygen species sequestration with or without biodegradation, incorporation and/or release of therapeutic agents including hormones and antimicrobial agents, and selective incorporation of linking moieties for target structure-property relationships (urea, urethane, thiourethane, sulfide, ester, carbonate, or amide). Device application examples are proposed to demonstrate the system processing flexibility across many fabrication techniques for medical devices including melt processing (extrusion, injection molding, film blowing, fused deposition modeling), solvent processing (dip coating, evaporative film casting, electrospinning), thermoset processing (reactive injection molding, gas blown foaming due to carbon dioxide reaction products when reacting an isocyanate group with water or a carboxylic acid monomer, reactive film casting, UV curing including stereolithography), porous templating (high internal phase emulsion templating, supercritical gas blowing, porogen templating, 3D printing).

### Definitions

Aliphatic: An organic compound (such as an alkane) having an open-chain structure.

Biodegradable: A polymeric material that can be eroded and removed from a biological system under biologic conditions through a combination of chain scission that reduces molecular weight and/or crosslink density, dissolution, and endocytosis.

Biodegradation: degradation due to the biological environment (can be modeled by in-vitro tests).

Bioresorption: process by which a biomaterial is degraded in the physiological environment and the product(s) eliminated and/or absorbed.

Biodurable: a polymeric material that maintains molecular weight, crosslink density, and does not undergo appreciable mass loss when exposed to biologic conditions.

Branched Monomer: A reactive monomer used for polymer synthesis with >2 reactive functionalities. This monomer is used as a crosslinker, either in the initial polymer synthesis, or during a subsequent crosslinking step.

Catalyst, Catalyzation: A substance that increases the rate of a chemical reaction without itself undergoing any permanent chemical change.

Chain Extender: A low molecular weight (MW) reagent that converts polymeric precursors to higher molecular weight derivatives.

Crosslink: The process of forming physical, covalent bonds, or relatively short sequences of chemical bonds to join two polymer chains together.

Crosslinker: A molecule that contains two or more reactive ends capable or chemically attaching to specific functional groups of multiple molecules to bind them together.

Crosslinking sensitizer: A molecule that increases the likelihood of a polymer system forming a covalent crosslink when exposed to an external energy source through increased reactivity and/or increased molecular mobility.

Curable: the ability of a polymer, prepolymer, macromer, or monomers to solidify, toughen, or harden a liquid resin, or polymeric material by reacting monomers or cross-linking polymer chains, brought about by ionizing radiation, UV radiation, heat, or chemical reactivity.

Depot Injection: an injectable medication formulation with an extended-release profile, requiring a lower frequency of administration. Examples include an injection that is liquid at room temperature and subsequently gels at body temperature for increased stability in-vivo.

Elastomer: a polymer with viscoelasticity (i.e., both viscosity and elasticity) and with weak intermolecular forces, generally low Young's modulus (E) and high failure strain compared with other materials.

Equivalent: a reactive group on a molecule. For example, 1 mole of the difunctional molecule N-methyl diethanolamine has 2 moles of reactive OH equivalents.

Glassy Modulus: the elastic modulus of a material below the glass transition temperature. The ratio of the force on a material to the resulting elastic deformation.

Hard Segment: the portion of a block copolymer repeat unit that contributes to the rigid polymer phase. Typically composed of a diisocyanate and chain extender.

Initiator: a source of any chemical species that reacts with a monomer to form an intermediate compound capable of linking successively with a large number of other monomers into a polymeric compound. For example: a molecule capable of forming a free radical to initiative a radical polymerization.

Linear Monomer: a reactive monomer used for polymer synthesis with 2 reactive functional groups that participate in the polymerization reaction. Additional reactive groups can be present on the monomer, but these do not participate in the polymerization reaction.

Macromer: a macromolecule with at least one end-group that enables it to act as a monomer and contributes a single monomeric unit to the polymer chain.

Melt processing: manufacturing methods that use the liquid rheology properties of molten thermoplastic polymers that have been heated above the melt transition temperature.

Network: A highly ramified macromolecule in which essentially each constitutional unit is connected to each other constitutional unit and to the macroscopic phase boundary by many permanent paths through the macromolecule, the number of such paths increasing with the average number of intervening bonds; the paths must on the average be co-extensive with the macromolecule.

Oligomer: Oligomers are low molecular weight polymers comprising a small number of repeat units whose physical properties are significantly dependent on the length of the chain.

Plasticized: To make or become plastic, as by the addition of a plasticizer.

Rubbery Modulus: The elastic modulus of a material above the glass transition temperature. The measure of the elastic energy stored in the material above the glass transition temperature.

Soft Segment: the portion of a block copolymer repeat unit that contributes to the flexible polymer phase. Typically composed of a polyether or polyester segment.

Solvent Processing: manufacturing methods that use the liquid rheology properties of thermoplastic polymers dissolved into solution with an appropriate solvent.

Stereolithography: A technique or process for creating three-dimensional objects, in which a computer-controlled moving laser beam is used to build up the required structure, layer by layer, from a liquid polymer that hardens on contact with laser light.

Supercritical gas: Any substance at a temperature and pressure above its critical point, where distinct liquid and gas phases do not exist, but below the pressure required to compress it into a solid.

Sulfide: an organosulfur functional group with connectivity of R—S—R′. Also known as a thioether.

Thermoplastic: A plastic polymer material that becomes pliable or moldable at a certain elevated temperature and solidifies upon cooling.

Thermoset: A polymer that is obtained by irreversibly hardening, or curing, a soft solid or viscous liquid prepolymer.

### Reactive Oxygen Species Labile Material Platform

Many traditional biodegradable polymers undergo hydrolytic degradation (Polyesters including Polycaprolactone, Polylactic acid, Polyglycolic acid; polycarbonates; polyanhydrides). These materials lose molecular weight when exposed to an aqueous in-vivo environment. While useful, these materials have a few drawbacks for tissue engineering applications. The degradation products of most hydrolytic systems are acidic. These degradation products lower the pH of the local tissue, and provide acid-based catalysis of the remaining material to expedite further degradation and acidic byproduct release in a forward feedback loop. In some cases, this rapid mass loss can lead to device failure modes such as imbalanced force transfer and late-stage acid-induced tissue necrosis. Rapid hydrolysis can be mitigated by using a hydrophobic material system that restricts degradation to the surface of the material (instead of bulk degradation of a hydrophilic hydrolytic material). However, even surface degrading hydrolytic systems have limitations in tissue engineering applications by responding less to cellular activity, and more to ambient tissue conditions. These interstitial tissue conditions (such as localized pH) can vary between patients and have inherent variation in material degradation characteristics. Conversely, oxidatively degrading biomaterial systems decrease molecular weight in response to reactive oxygen species secreted by the body, which are predominantly immune cells coordinating a healing response. Oxidative degradation enables higher spatiotemporal control of decreased molecular weight and clearance from the tissue site that is matched with the localized tissue response to an implant. This dovetailed degradation and tissue remodeling relationship enables improved patient-specific force transfer and tissue integration. These interactions help mitigate device failure modes associated with premature mass loss and device weaking, including device fracture, mechanical failure, loss of sealing, loss of tissue integration, recanalization, and material particulation prior to tissue integration. Compared to hydrolytic systems, oxidative degradation products do not self-catalyze further degradation, enabling further control of the mass loss profile in-vivo. Embodiments provide a flexible biomaterial platform that enables oxidative degradation of implants through the facile incorporation of oxidatively labile tertiary amine functionalities. These motifs inherently sequester reactive oxygen species (ROS) through the degradation pathway. Additional ROS sequestration is proposed in other embodiments that include sulfide linkages (R—S—R) in addition to the labile tertiary amines. The use of di-functional monomers that contain tertiary amines for targeted oxidative degradation is an important delineation from prior art that uses branched tertiary amine monomers with a functionality greater than 2. Difunctional monomers impart greater flexibility in the design of the polymer network, enabling selective degradation between crosslinks in a thermoset network. Difunctional monomers also enable thermoplastic polyurethanes that are inherently incompatible with polyols with a functionality greater than 2. These attributes allow for independent control of the polymer network crosslink density and molecular weight of degradation products that occur between covalent crosslinks in a thermoset system. It also allows control of the degradation product molecular weight within a thermoplastic repeat unit. As an example, an embodiment may create a hydrolytically stable polyurethane elastomer with low crosslinking, but terminal biodegradation products that have a lower molecular weight than the molecular weight between crosslinks.

Embodiments Using Condensation Reactions with Isocyanates:

### Thermoplastic Embodiments:

A polymer polymerized from monomers consisting of at least one diisocyanate (aliphatic or aromatic) and at least one diol containing a tertiary amine in the chain backbone (examples include, but are not limited to, N-Methyldiethanolamine, N-tert-Butyldiethanolamine,1,4-Bis(2-hydroxyethyl)piperazine, N-Ethyldiethanolamine, avridine, N-butyldiethanolamine, 2,2′-(Octadecylimino)diethanol, N,N-Bis(2-hydroxyethyl)dodecylamine). Aromatic tertiary amine diols such as N,N-bis(2-Hydroxypropyl)aniline are consciously avoided to mitigate the potential formation of carcinogenic aromatic amines during biodegradation. Aliphatic diisocyanate examples include 1,6-Hexamethylene diisocyanate, 2,2,4-Trimethyl-1,6-hexamethylene diisocyanate (TMHDI), isophorone diisocyanate, 4,4′-Dicyclohexylmethane diisocyanate, 1,4-Cyclohexylene diisocyanate, L-Lysine ethyl ester diisocyanate. The tertiary amine linkage can be present in the hard or soft segment of the polymer. Incorporating a tertiary amine linkage in the backbone of the thermoplastic repeat unit enables polymer degradation in an oxidative environment, such as in the human body. When exposed to reactive oxygen species, the tertiary amines in the polymer are oxidized into a population of secondary amines and degradation oligomers functionalized with carboxylic acid, primary amine, and aldehyde end groups. When used as a biomaterial, this polymer will biodegrade in the presence of ROS (reactive oxygen species) produced by cells according to the natural immune response. This oxidation will not only occur via cell mediated immune and healing responses, but also by neutralizing environmental ROS. This localized ROS scavenging is an added benefit to the device by mitigating factors that contribute to chronic inflammation. This is advantageous for implants in high ROS tissue environments, such as diabetic ulcers and other diabetic maladies in peripheral tissues, such as Charcot foot. The oxidative mechanism also has the benefit of limiting self-catalyzation seen with hydrolytic systems, where acidic hydrolysis byproducts lower local pH levels and contribute to further, more accelerated hydrolysis.

Other embodiments include tertiary amine containing monomers terminated with hydroxyl and/or an amine (primary or secondary) functional group to form urethane and urea linkages, respectively, when reacted with isocyanates. The total functionality for any combination of hydroxyl and primary amines for the degradable tertiary amine containing monomer would be two for a thermoplastic system, and greater than two for thermoset systems. Example bifunctional monomers with a degradable tertiary amine linkage in the backbone include, but are not limited to, N,N-Bis[3-(methylamino)propyl]methylamine, 3,3′-Diamino-N-methyldipropylamine, 1,4-Bis(3-aminopropyl)piperazine, 1-[2-(2-Hydroxyethoxy)ethyl]piperazine, 1-Amino-4-(2-hydroxyethyl)piperazine, 1,4′-Bipiperidin-3-ol. Other diol, diamine, dithiol (or other difunctional amine, alcohol, thiol) chain extenders can be added to the synthesis to impart other functional properties in addition to oxidative degradation (e.g., controlling Tg, hydrophobicity, toughness, crystallinity, melt transition temperature, elastic modulus, etc.). Diamine terminated chain extension examples include, but are not limited to, 1,3-Diaminopentane; 1,3-Diamino-2-propanol; 1,5-Diamino-2-methylpentane; Hexamethylenediamine; 2,2-Dimethyl-1,3-propanediamine; 1,3-Diaminopropane; 4,9-Dioxa-1,12-dodecanediamine; 1,3-Cyclohexanebis(methylamine); and Ethylenediamine. Diol terminated chain extension examples include, but are not limited to; ether containing diols such as diethelyene glycol, triethylene glycol, tetraethylene glycol; ester containing diols such as polycaprolactone diol; hydrolytically labile polyesters including polylactic acid, polyglycolic acid, polylactic-co-glycolic acid; other oxidatively susceptible or labile linkages including difunctional thioethers, difunctional ethers; aliphatic linear diols such as Butane-1,3-diol; 1,4-Pentanediol; 1,5-Hexanediol; 2,4-Pentanediol; 1,5-Pentanediol; 2,5-Hexanediol; 1,6-Hexanediol; 1,4-Butanediol; 1,3-propanediol; 3-Methyl-1,5-pentanediol; 2,2-Dimethyl-1,3-propanediol; 2-Butyl-2-ethyl-1,3-propanediol; ring containing diols such as 1,4-Cyclohexanediol; 1,4-Cyclohexanedimethanol; sulfide containing diols such as 1,4-Dithiane-2,5-diol; 3,6-Dithia-1,8-octanediol, 2,2′-thiodiethanol, 3,3′-Thiodipropanol, 2-hydroxyethyl disulfide; diols with ether pendant groups such as 3-propoxypropane-1,2-diol; 3-Ethoxy-1,2-propanediol; 3-Allyloxy-1,2-propanediol; and trimethylolpropane allyl ether; unsaturated diols such as 5-Norbornene-2,2-dimethanol; 3-4-Dihydroxy-1-butene; 1,5-Hexadiene-3,4-diol; 4-cyclopentene-1,3-diol; 7-Octene-1,2-diol; and fluorinated diols such as 2,2,3,3,4,4-Hexafluoro-1,5-pentanediol; 2,2,3,3,4,4,5,5-Octafluoro-1,6-hexanediol; 1H, 1H, 4H, 4H-Perfluoro-1,4-butanediol; Fluorinated tetraethylene glycol; and fluorinated triethylene glycol.

Many embodiments contemplate aliphatic systems, including aliphatic diisocyanate monomers, but other contemplate aromatic monomers, including aromatic diisocyanates (2,4-Toluene diisocyanate, 2,6-Toluene diisocyanate, p-Phenylene diisocyanate, m-Xylylene diisocyanate, m-Tetramethylxylene diisocyanate, 4,4′-diphyylmethane diisocyanate, 1,5-Napthalene diisocyanate) and aromatic diols, for biomedical applications that require higher toughness and higher material modulus, such as orthopedic implants. When using aromatic monomers, aliphatic chain extenders should be used between aromatic motifs and the tertiary amine degradation site. This is to prevent small molecular weight aromatic diamines as terminal degradation products.

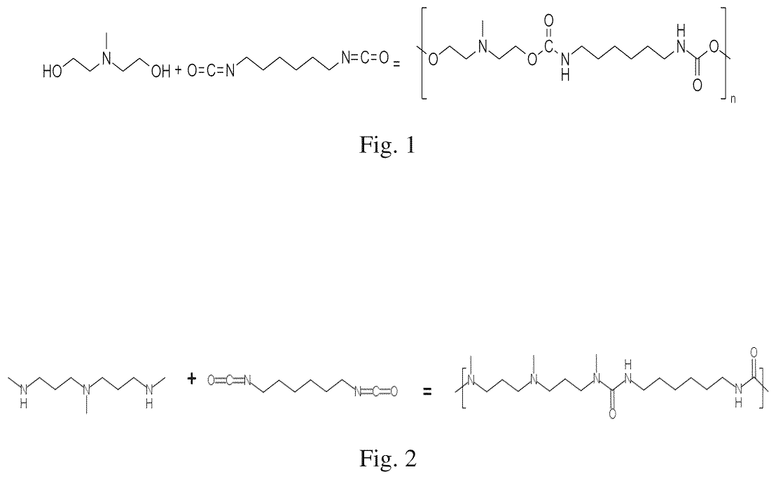

FIG. 1 depicts an embodiment of a tertiary amine polyurethane synthesis.

In an alternative embodiment, the reactive hydroxyl group can be replaced with a secondary amine to form a tertiary amine containing polyurea when reacted with a diisocyanate. For example, FIG. 2 depicts an embodiment of a tertiary amine polyurea synthesis.

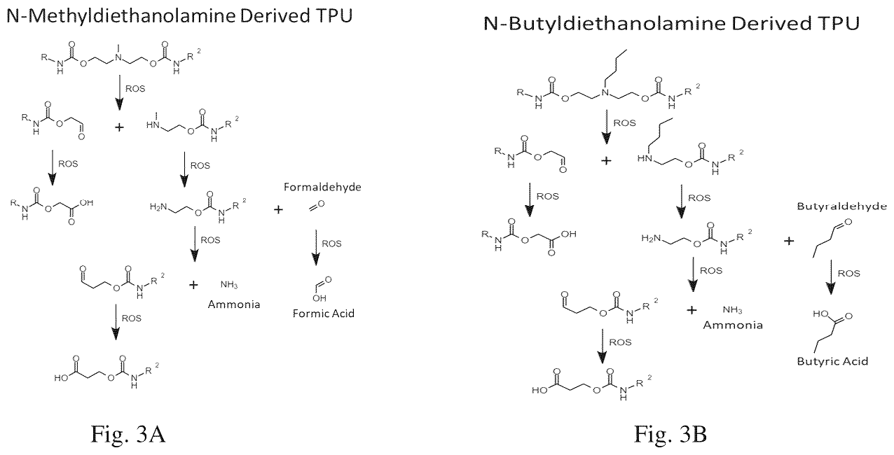

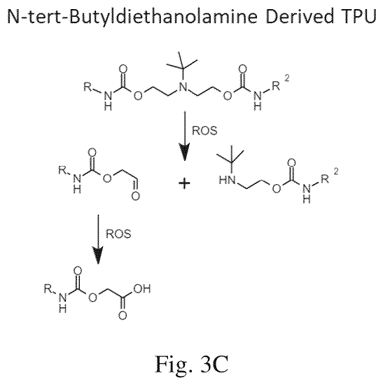

FIGS. 3A, 3B, 3C depict embodiments of degradable repeat unit oxidation pathways.

A thermoplastic embodiment has the advantage of incorporating tertiary amines into the thermoplastic repeat unit for the desired oxidative degradation response while still maintaining the benefits of thermoplastic polymer manufacturing flexibility (melt processing, solvent casting, etc.).Polyurea content can be increased in the polymer backbone to increase hydrogen bonding and yield high modulus formulations for high load bearing biomedical applications such as orthopedic implants, or materials that resist creep for tendon or ligament fixation applications.

Other thermoplastic embodiments include aliphatic diols with a tertiary amine in the backbone and terminal alkene functionalities such as 2,2′-[1,2-Ethanediylbis(2-propenylimino)]bisethanol; 2-[Allyl(2-hydroxyethyl)amnio]ethan-1-ol; and N-Allyl-2,2′-Iminodiethanol. These thermoplastics have pendant alkene groups branching from tertiary amines in the polymer backbone. After melt processing these parts into a finished geometry, these pendant alkene groups can be polymerized together with radical initiation to form a crosslinked thermoset. Radical initiation can derive from ionizing radiation, thermal initiators, redox initiators, or UV initiators. The resulting thermoset polymer is biodegradable at the tertiary amines that form the covalent crosslinks of the network.

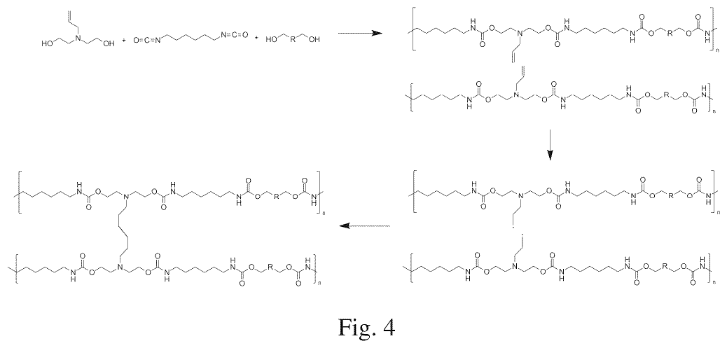

FIG. 4 depicts an embodiment of a (a) crosslinkable, oxidatively degradable tertiary amine containing thermoplastic, (b) radical formation due to ionizing radiation exposure, and (c) direct crosslinking of thermoplastic pendant groups.

To increase radical crosslinking sensitivity, or impart other moieties into the polymer network, crosslinking sensitizers can be included in phase with the thermoplastic polymer. Radiation sensitizer examples are difunctional or polyfunctional molecules terminated with unsaturated carbon to carbon bonds (alkenes, alkynes, acrylates, methacrylates, alkynes, norbornenes, etc.). Sulfide containing radiation sensitizers for ROS sequestration include Bis(2-methacrylolyl)oxyethyl disulfide, N,N′-Bis(acryloyl)cystamine, and allyl disulfide. Crosslink sensitizer molecules can be synthesized from targeted therapeutic molecules for in-vivo release after implantation. For example, estradiol can be esterified with an alkene terminated carboxylic acid (2-Carboxyethyl acrylate, mono-2-(Methacryloyloxy)ethyl succinate, 3-Butenoic acid, 4-Pentenoic acid, Allylglycine, Acrylic acid, Methacrylic acid) to yield a radiation crosslinkable di-alkene terminated estradiol molecule. The sensitizing molecule is included in phase with a thermoplastic part and crosslinked with the thermoplastic network using ionizing radiation, potentially during routine medical device sterilization using Electron beam or Xray sterilization. After implantation of the material, hydrolysis of the ester linkages would liberate bioactive estradiol as a function of network crosslink scission. The residual implant thermoplastic repeat unit could undergo oxidative degradation at the tertiary amine, which is a separate mechanism from the release of the therapeutic molecule. This therapeutic agent would be well suited for orthopedic implants or treatments for patients with osteoporosis.

Alternatively, crosslinking agents or crosslinking sensitizers can include difunctional or polyfunctional thiol terminated molecules. These molecules are included in phase with the thermoplastic polymer and crosslink via thiol-ene click chemistry. Thiol crosslinking agents include, but are not limited to, ethylene glycol bis-mercaptoacetate, butane dithiol, 3-mercapto-1,2-propanediol, 1-thioglycerol, DL-Dithiothreitol (DTT), 2-hydroxymethyl-2-methyl-1,3-propanediol tris-(3-mercaptopropionate), Pentaerythritol tetrakis(3-mercaptopropionate) or Trimethylolpropane tris(3-mercaptopropionate). Polythiol crosslinkers without ester groups include Pentaerythrityl tetrathiol, Benzene-1,2,4,5-tetrathiol, 3,6-octane-1,3,6,8-tetrathiol, Propane-1,2,4 trithiol, 1,3,5-triazine-2,4,6-trithiol, Hexane-1,2,4 trithiol, and Tetra(2-mercaptoethyl)silane. Alternatively, target therapeutic agents can be modified into a thiol crosslinking agent for release after in-vivo implantation. As an example, estradiol could be esterified with a thiol terminated carboxylic acid (3-Mercaptopropionic acid, 4-mercaptobutyric acid, 6-Mercaptohexanoic acid, Thioglycolic acid, 2-Methyl-3-sulfanylpropanoic acid, 4-Mercaptobenzoic acid, Mercaptosuccinic acid, Thiolactic acid) yielding a dithiol terminated estradiol macromer. This thiol terminated estradiol macromer is included in phase with the thermoplastic polymer and crosslinked into the network using thiol-ene click chemistry. After implantation, hydrolysis of the ester linkages would liberate bioactive estradiol as a function of crosslink scission. The residual thermoplastic repeat unit could undergo oxidative degradation in a separate mechanism from the therapeutic release. Estradiol would be well suited for orthopedic implants or therapeutic treatments such as gelling depot injections for patients with osteoporosis.

Other bioactive agents that can be included in the crosslinking segment (via alkene, alkyne, or thiol termination) include, but are not limited to peptides with cysteine residues, amino acids (such as L-arginine), hydroxycinnamates (ferulic acid, caffeic acid, sinapic acid, p-coumaric acid, m-coumaric acid, L-Tyrosine), and other natural antioxidants (thiamine, lipoic acid, glutathione, daidzein, allicin, melatonin, and curcumin).

Alternative macromers can be synthesized to incorporate other functional properties into the polymer network while maintaining oxidative degradation capacity with tertiary amine, and additional ROS sequestration with sulfide linkages. Macromers can incorporate peptide sequences, mesogens, chromophores, or associative polymer repeat units.

Other therapeutic compounds not explicitly named can be modified at a variety of functional groups to become crosslinking agents, provided the new terminal functional groups of the modified therapeutic agent interact with the thermoplastic network to form a crosslink (i.e., terminal thiols or alkene groups to crosslink alkene pendant groups on the thermoplastic). Therapeutic agents with terminal hydroxyl groups can be esterified with carboxylic acids (3-mercaptoproprionic acid, 4-Pentenoic acid) or with acyl halides (acryloyl chloride, methacryloyl chloride) in the presence of a base catalyst (triethylamine) to form hydrolytically labile ester linkages, modified with chloroformates to form hydrolytically labile carbonate linkages (Allyl chloroformate, vinyl chloroformate), or functionalized with biostable urethane linkages using isocyanates. Therapeutic agents with terminal thiols (such as cysteine residues on peptides) can be functionalized with allyl, vinyl, methacrylate, acrylate, or norbornene modifiers (3-Methyl-3-buten-1-ol, 4,Penten-2-ol, 1,6-heptadien-4-ol, 1,5-Hexadien-3-ol, 3-Buten-1-ol, 4-Penten-1-ol, 5-Hexen-1-ol, 9-Decen-1-ol, 10-Undecen-1-ol, Allyl alcohol, 5-Norbornen-2-methanol). Therapeutic agents with terminal primary amines can be functionalized into ureas with isocyanate terminated modifiers and functionalized into amides with carboxylic acid terminated modifiers. Therapeutic agents with terminal carboxylic acids can be functionalized into amides with isocyanate modifiers and functionalized into hydrolytically labile esters with hydroxyl terminated modifiers (2-Hydroxyethyl methacrylate, 4-Hydroxybutyl acrylate, 2-Hydroxyethyl acrylate, 3-Mercapto-1-propanol, 6-mercapto-1-hexanol, 8-Mercapto-1-octanol, 9-Mercapto-1-nonanol, 11-Mercapto-1-undecanol, 2-mercaptoethanol, 1-Mercapto-1-propanol, 3-Mercapto-3-methylbutan-1-ol, 2-mercapto-3-butanol, 3-Mercapto-1-hexanol).

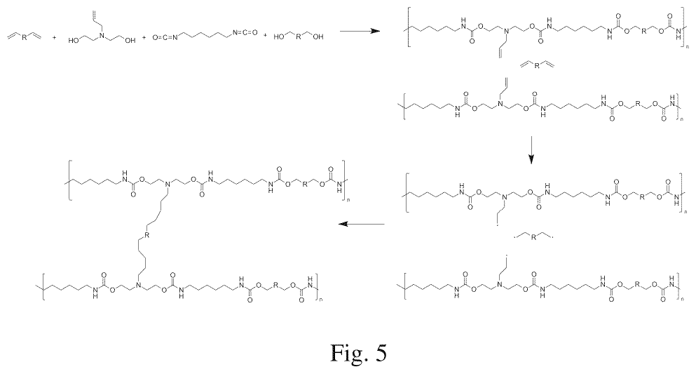

FIG. 5 depicts an embodiment of (a) a crosslinkable, oxidatively degradable tertiary amine containing thermoplastic, (b) radical formation due to ionizing radiation exposure via radiation sensitizing agents, and (c) crosslinking with a reactive macromer.

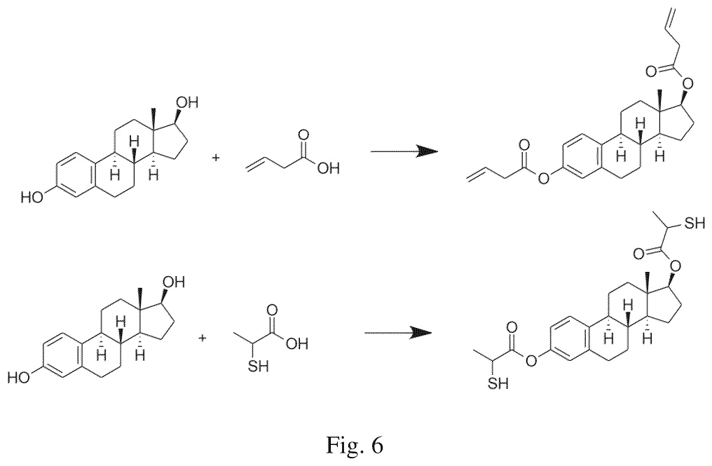

FIG. 6: Formation of therapeutic agent macromers. The top formation shows a radiation sensitizing crosslinker formed from estradiol. The bottom formation shows a thiol-ene crosslinking agent formed from estradiol. Both can be used to crosslink pendant alkene groups when included in phase with a thermoplastic part and exposed to an external energy source (Ultraviolet light or ionizing radiation). These macromers can also be used for direct polymerization into thermoplastics or thermosets.

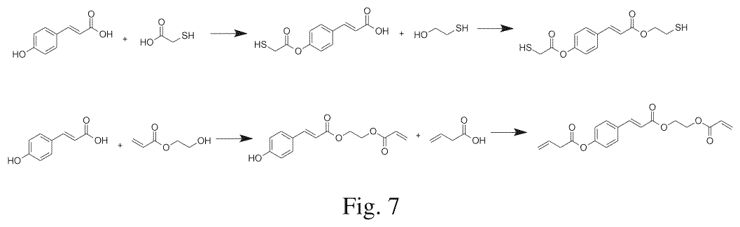

FIG. 7 depicts embodiments of additional therapeutic agent macromers for incorporating antimicrobial p-coumaric acid into a polymer network. Either as a crosslinking agent between pendant thermoplastic alkene groups, or via direct polymerization.

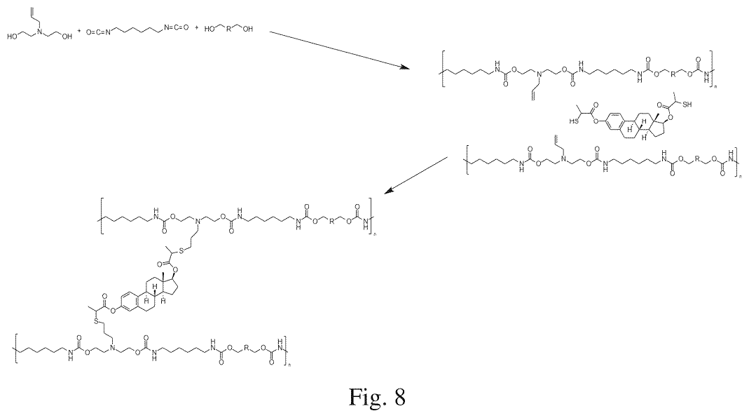

FIG. 8 depicts an embodiment of covalent crosslinking of polyurethane thermoplastic chains using a thiol terminated estradiol macromer.

Other thermoplastic embodiments employ chain extension of a polymer network using tertiary amine containing dicarboxylic acid monomers such as Methyliminodiacetic acid. In this embodiment, reactions between the carboxylic acid end groups and the reactive isocyanate in a diisocyanate or polyisocyanate form amide linkages and carbon dioxide gas. This monomer and other carboxylic acid terminated compounds can serve as reactive monomers and a chemical blow agent in forming gas blown foams as tissue scaffolds, in addition to traditional water blown foam systems yielding polyurethane-ureas.

Typical thermoplastic synthetic pathways include the reaction between a non-isocyanate monomer (diol, thiol, amine, etc.) and an isocyanate in stoichiometric reactive equivalent ratios ranging from 0.5:1 to 2:1 and, in an embodiment, 1:1.01, respectively. The final polymer network can result from either bulk reaction or polymerization in polar solvents (e.g., DMF, THF, DMSO). In systems of high hydrogen bonding or hard segment content>30%, the use of hydrogen bonding disruptors such as 2-propanol or salts can be employed to yield appropriate molecular weights. Reactions can be catalyzed by using catalysts containing tin, tertiary amines, or zinc and the combination thereof in amounts of 0.005 wt % to 5 wt %. Tertiary amine containing monomers in the thermoplastic synthesis can also double as catalysts for the synthesis. Using the tertiary amine containing monomer as the catalyst (referred here as “catalyst free”) has a distinct benefit for biomedical applications because the polymerized thermoplastic resin can be directly pelletized for subsequent melt processing into implantable devices without purifying the resin to remove residual tertiary amine catalysts (non-functionalized, such as DABCO), tin catalysts (stannous or stannic), or other catalysts that could negatively affect biocompatibility. As an example, a diisocyanate (aliphatic or aromatic) could be combined with a dried diol blend that includes at least one tertiary amine containing diol such as MDEA. The isocyanate and diol phases are mutually miscible without the use of any solvents and spontaneously react as function of tertiary amine content (i.e. increasing tertiary amine diol content increases reaction rate) in the presence of heat (for example, 50-1000 for 4-48 hours). The tertiary amine containing diol is consumed in the polymerization reaction and is incorporated into the polymer network without leaving residual tertiary amine monomer or other traditional catalysts used in polyurethane manufacturing. The resulting thermoplastic is medical implant ready without any post processing or purification to remove non-biocompatible catalysts. Additionally, the proposed example proceeds without any organic solvents for miscibility that could also negatively impact biocompatibility without removal (evaporation) during the manufacturing process and prior to implantation. This catalyst and solvent free manufacturing approach enables facile, low-cost manufacturing of oxidatively labile, bioresorbable thermoplastic polyurethanes. A similar approach can be employed using tertiary amine monomers in the catalyst free manufacture of thermosets without the use of catalyst for biomedical applications.

Beyond catalyst-free manufacturing of polymers, this approach can also enable in-situ curing of polymers as medical implants. For example, a tertiary amine containing monomer or macromer can be mixed with an isocyanate (aliphatic or aromatic) prepolymer, macromer, or monomer as a putty or liquid system to create a moldable or flowable two-part medical implant material system that reacts quickly without the use of harmful catalysts. This approach has utility in orthopedic applications such as critical sized bone defects, bone voids. Additionally, this approach could be used as a surgical sealant or hemostatic agent.

Solvent free, catalyst free, low-cost thermoplastic polymer feedstocks that oxidatively degrade via tertiary amine content (Tert Amine TPU's) have utility for biomedical implants, but also commodity plastic applications such as packaging, films, textiles, single use plastics, and other commercial applications. Once these commodity plastic parts enter the waste stream, the oxidatively labile nature of the polymer enable them to break down in biologic systems (including fish, animals, and human beings) and reduce the burden of micro or nanoplastics in the environment. As an example, a tertiary amine diol containing polyurethane thermoplastic can be synthesized as a low-cost resin and injection molded into fluid containers (e.g. packaging for beverages or other non-consumable daily use liquids) that are moisture stable to prevent negative consumer experiences or reduced shelf life, but still have a reduced lifespan before terminal degradation in the environment. If this class of packaging makes it into biologic systems in the form of microplastics, it can be broken down and cleared from the body via reactive oxygen species produced by immune cells. Other biodurable microplastics (not tertiary amine containing polyurethane thermoplastics) can induce oxidative stress. Tert amine TPU microplastics can serve as a buffer system to reduce the oxidative stress induced by other microplastics in biologic systems.

Solvent free, catalyst free, low-cost thermoset polymer feedstocks have utility for biomedical implants, but also commodity plastic applications such as sponges, foams, cushions, shoe soles, coatings, and other commercial applications. Once these plastic parts enter the waste stream, the oxidatively labile nature of the polymer enable them to break down in biologic systems (including fish, animals, and human beings) and reduce the burden of micro or nanoplastics in the environment. As an example, a oxidatively degradable thermoset polyurethane sponge with a glass transition temperature between 10 and 60 degrees Celsius could be manufactured as a household cleaning tool that ultimately breaks down in the environment when exposed to oxidative conditions, thereby reducing plastic waste. Environmental oxidative conditions include but are not limited to: reactive oxygen species formed in conjunction with exposure to UV radiation in sunlight; reactive oxygen species formed from microbes in a composting or anaerobic environment; reactive oxygen species from biologic systems (i.e. immune cells) that have ingested polymer particulates.

An embodiment includes a method as follows. (1) Remove moisture from diol monomers by applying heat (40-1000) and vacuum (10-100 mtorr) until dry (4-48 hours). (2) Blend the diol monomers into a single hydroxyl phase. Include at least one tertiary amine containing diol to serve as the reaction catalyst. No other additives (such as solvent or catalyst) are necessary, but can still be added. (3) Add isocyanate monomer to a closed jacketed reactor with impeller mixing and an inert atmosphere (nitrogen or vacuum). (4) Slowly add the hydroxyl phase to the isocyanate phase, using the reactor jacket to remove reaction exotherm and maintain the reaction below a threshold temperature (40-1000). (5) Add the hydroxyl phase until the NCO:OH equivalents are balanced to a target stoichiometric OH:NCO ratio between 1:0.9 to 1:1.1. (6) After complete addition of the hydroxyl phase, maintain mixing and keep the reaction below the threshold temperature as the reaction builds molecular weight into a prepolymer. (7) Dispense the thermoplastic prepolymer from the reactor into curing molds. (8) Cure the molds in an inert oven (vacuum or nitrogen atmosphere) to build the final thermoplastic molecular weight (40-100 C for 4-96 hours).

### Example Formulations:

| | | | Equivalent Ratio |
|---|---|---|---|
| NCO | Diol 1 | Diol 2 | (NCO:Diol 1 OH:Diol 2 OH) |
| TMHDI | MDEA | HDO | 3:1:2 |
| TMHDI | MDEA | HDO | 10:9:1 |
| TMHDI | MDEA | HDO | 10:1:9 |
| TMHDI | MDEA | TDO | 3:1:2 |
| TMHDI | MDEA | TDO | 10:9:1 |
| TMHDI | MDEA | TDO | 10:1:9 |
| HDI | MDEA | HDO | 3:1:2 |
| HDI | MDEA | HDO | 10:9:1 |
| HDI | MDEA | HDO | 10:1:9 |
| HDI | MDEA | TDO | 3:1:2 |
| HDI | MDEA | TDO | 10:9:1 |
| HDI | MDEA | TDO | 10:1:9 |

### Monomer Acronym References are:

| Acronym | Monomer Name | Molecular Weight |
|---|---|---|
| TMHDI | Trimethyl hexamethylene diisocyanate | 210.28 |
| MDEA | N-Methyldiethanolamine | 119.16 |
| HDO | 1,6-Hexanediol | 118.17 |
| TDO | Thiodiglycol | 122.19 |
| HDI | Hexamethylene diisocyanate | 168.2 |

Residual catalyst in the polymer system can be identified by, for example, extracting a polymer component with a solvent to remove the mobile catalyst from the polymer network and analyzing the extract with Liquid Chromatography Mass Spectrometry (LCMS) for small organic catalyst molecules such as tertiary amine catalysts, or by using Inductively coupled plasma mass spectrometry (ICPMS) to identify metallic ions associated with organometallic catalysts such as tin catalysts.

Other embodiments include poloxamer-like triblock polymers with thermal gelation properties. These triblock polymer embodiments have a hydrophobic core chain with hydrophilic chains on either end. A distinction between this embodiment and conventional thermal gelation poloxamer compositions is the ability to sequester ROS (due to sulfide linkages, for example) and/or degrade in the presence of ROS (due to tertiary amines, for example). Hydrophobic core repeat units can be derived from propylene glycol; methylated repeat units derived from ring opening polymerizations of propylene oxide; polysulfide repeat units; repeat units derived from thiol terminated sulfides such as 2,2′-Thiodiethanethiol; polyfumaric acid repeat units; polyisobutylene repeat units; or polyisoprene repeat units. Hydrophilic flanking chains in the triblock polymer can include repeat units of ethylene oxide; repeat units derived from thiol terminated polyethers such as Tetra(ethylene glycol) dithiol; polysulfoxides; polysulfones; oxide repeat units derived from polysulfide diols such as 3,6-Dithia-1,8-octanediol; methylated tertiary amine containing repeat units derived from N-Methyldiethanolamine, N,N-Bis[3-(methylamino)propyl]methylamine, or 3,3′-Diamino-N-methyldipropylamine; butylated repeat units derived from N-tert-butyldiethanolamine or N-Butyldiethanolamine; or tertiary amine containing repeat units resulting from the ring opening copolymerization of an epoxide (including but not limited to propylene oxide or ethylene oxide) and a secondary amine (including but not limited to dimethylamine, diethylamine, dibutylamine, diisopropylamine, 2-(Ethylamino)ethanol, 2-(Methylamino)ethanol, 3-Methylamino-1-propanol, 2-(butylamino)ethanol, piperidine, or piperazine). In any combination, the triblock copolymer can sequester ROS through sulfide linkages or ROS labile tertiary amines through oxidative degradation. Hydrophobic chains can be linked to hydrophilic chains through thioether, urea, urethane, thiourethane, carbonate, ester, or ether linkages. Some embodiments can include two selections from the provided list of hydrophilic chains or provided list of hydrophobic chains, as long as the two chain selections have differential hydrophobicity relative to each other (log P) to drive thermal gelation interactions. Thiol-ene chain grown termination can be achieved with Acrylate-PEG-NHS, Diethylene glycol monoallyl ether, and cysteamine. Condensation polymerization chain growth with diisocyanates can be terminated with monomers containing one NCO reactive group such as allyl alcohol, allyl amine, and diethylene glycol monoallyl ether. Ring opening polymerization of lactams such as caprolactam can be employed to yield amide linkages between hydrophilic and hydrophobic segments.

In one embodiment, a thiol-ene macromer or pre-polymer system is chain terminated using cysteamine by reacting the thiol functional group with alkene(s) or alkyl halide at the end of the molecule/macromer/oligomer, resulting in primary amine termination. This amine functionality can be leveraged for subsequent reactions with isocyanate monomers.

In some embodiments, the oxidatively active segments of the triblock copolymer serve as a carrier for therapeutic agents in a depot injection. As functional groups within the polymer are oxidized (such as tertiary amines or sulfide linkages) by the in-vivo environment, the localized decreased in polymer hydrophobicity leads to unfavorable solubility of the drug and release into the tissue environment. For example, sulfides are converted to sulfoxides, and sulfones under oxidative environments. This oxidation behavior can occur in conjunction with, or independent of oxidation degradation of the triblock copolymer.

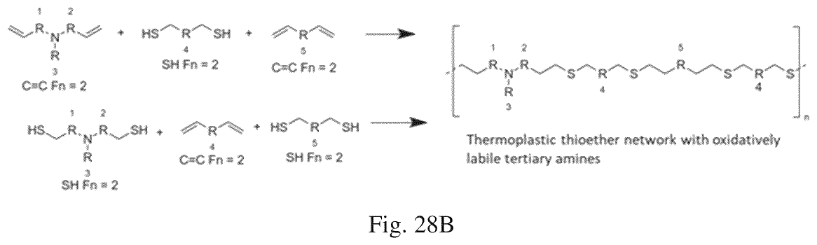

FIG. 28A depicts a monomer scheme to synthesize a thermoplastic thioether with tertiary amine functional groups for oxidative sequestration and oxidative degradation in-vivo. FIG. 28A shows two possible routes to arrive at a thermoplastic thioether network with oxidatively labile tertiary amines. FIG. 28B depicts an illustrate example of the scheme of FIG. 28A. FIG. 28B shows a thermoplastic thioether network with oxidatively labile tertiary amines that may be generated via either path of FIG. 28B.

### Thermoset Embodiments:

Examples of degradable, tertiary amine containing, multifunctional crosslinkers include: Polyamines including tris(2-aminoethyl)amine; N,N,N′,N′-tetrakis(2-hydroxyethyl)ethylenediamine; tris(3-aminopropyl)amine; tris[2-(methylamino)ethyl]amine; N,N,N′,N′-tetrakis(3-aminopropyl)-1,4-butanediamine; and polyols including triethanolamine; N,N,N′,N′-Tetrakis(2-hydroxypropyl)ethylenediamine; N,N,N′,N′-Tetrakis(2-hydroxyethyl)ethylenediamine; Triisopropanolamine; and Tripropanolamine. Tertiary amine containing thiols include Tris(2-mercaptoethyl)-amine and 2,2′-(methylimino)diethanol. Other polyfunctional tertiary amine crosslinkers include N,N-Bis(2-hydroxyethyl)glycine and N-(2-Hydroxyethyl)iminodiacetic acid. These crosslinkers can be combined with diisocyanates, polyisocyanates, branched polyols, branched polythiols, diol chain extenders, thiol chain extenders, and amine chain extenders to form a thermoset network. Degradation would occur at the tertiary amine crosslinking sites, and at any additional tertiary amine functionalities included in the chain extenders.

Examples of polyol crosslinkers include glycerol, pentaerythritol, 1,2,4-butanetriol, 1,2,6-Hexanetriol, 1,1,1-Tris(hydroxymethyl)propane, and Di(trimethylolpropane). These aliphatic polyol crosslinkers are biostable and would need to combine with tertiary amine chain extenders or crosslinkers to enable a biodegradable thermoset system when reacted with diisocyanates or polyisocyanates.

### Radical Polymerizations Embodiments:

### Branched Macromer Embodiments:

In an alternative embodiment, the tertiary amine containing monomer (hydroxyl, secondary amine, primary amine, thiol or carboxylic acid terminated) could be reacted with an a) diisocyanate or polyisocyanate and b) an alkene (C═C) terminated monomer that also reacts with the isocyanate, such as, but not limited to, allyl alcohol, allylamine, N-allylmethylamine, 3-Buten-1-amine, 2-methyl-2-propen-1-amine, or 3-butenoic acid for alkene end-capping of polyurethane macromer containing an oxidatively labile tertiary amine. Alternatively, alkene groups can be incorporated directly into the macromer backbone via unsaturated diols such as 1,4-butene diol, or as pendant alkene groups with monomers such as trimethylolpropane allyl ether. The reaction product would be alkene-functionalized polyurethane macromers (terminal alkene groups, backbone alkene groups, or pendant alkene groups) with oxidatively labile tertiary or secondary amine linkages in the polymer backbone. In the examples previously described, alkene functional groups can be replaced with alkyne functional groups for reaction with amines and thiols. Monomers for alkyne functionalization include propargyl alcohol, 2-butyn-1-ol, methyl propargyl ether, 2-butyne-1,4, diol, 3-butyn-2-ol, 3-Butyn-2-ol, propiolic acid, 2-Pentyn-1-ol, 2,4-Hexadiyne-1,6-diol, 2-methyl-3-butyn-2-ol, 3-Pentyn-2-ol, 2-hexyn-1-ol, 3-hexyn-2-ol, 5-Hexen-2-yn-1-ol, 2-heptyn-1-ol, 1-hexyn-3-ol. In the alkene examples previously described, alkene functional groups can be replaced with acrylate or methacrylate functional groups for radical polymerization or click chemistry with thiols. Acrylate and methacrylate monomers for functionalization include 2-hydroxyethyl methacrylate, mono-2-(methacryloyloxy)ethyl maleate, glycidyl methacrylate, 2-hydroxyethyl acrylate, hydroxypropyl acrylate, and glycerol 1,3-digrlycerolate diacrylate. These C═C functionalized macromers could be solution blended with multifunctional thiol monomers (such as pentaerythritol tetrakis(3-mercaptopropionate), trimethylolpropane tris(3-mercaptopropionate), or 2-hydroxymethyl-2-methyl-1,3-propanediol tris-(3-mercaptopropionate) to enable a UV crosslinkable thermoset resin in the presence of a photoinitiator or heat crosslinkable thermoset in the presence of a thermal initiator. The covalent crosslinks of the final thermoset would be from the thiol crosslinker, or a branched polyol, polyamine, or polycarboxylic acid added to the initial macromer synthesis. Examples of branched monomers to create branched macromers include pentaerythritol, hexanetriol, 1,1,1-Tris(hydroxymethyl)ethane, triethanolamine, or other multifunctional tertiary amines previously described. Additional multifunctional monomers (such as diols) can be added to change the functional properties of the macromer (i.e., solubility parameters, rheology, etc.) or the final thermoset (toughness, hydrophobicity, Tg, modulus etc.) This resin would be especially useful for stereolithography, inkjet printing, direct ink write printing, continuous liquid interface printing, or other UV additive manufacturing techniques, in addition to UV curable coatings for medical devices.

Linear macromers (liquid)+linear reactive monomers in solution=high molecular weight linear thermoplastic reaction product. Example: Allyl alcohol+hexamethylene diisocyanate+N-Methyldiethanolamine=linear macromer reaction product “A”; 2,2′-(Ethylenedioxy)diethanethiol=linear reactive monomer “B”. Combining A and B in the presence of a radical initiator (i.e., UV photoinitiator and UV stereolithography light source) would result in a thermoplastic polymerization.

Linear macromers (liquid)+branched crosslinker in solution=thermoset reaction product. Reaction of a liquid resin to form a solid polymer are especially useful for stereolithography 3D printing applications that require a biodegradable implant.

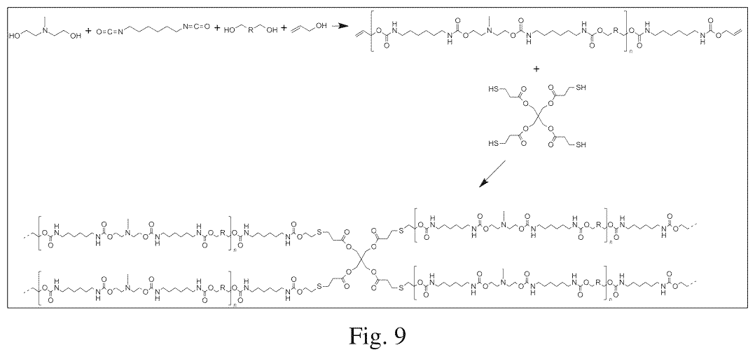

FIG. 9 depicts an embodiment of thermoset synthesis using a linear tertiary amine macromer with alkene termination combined with a polythiol crosslinker.

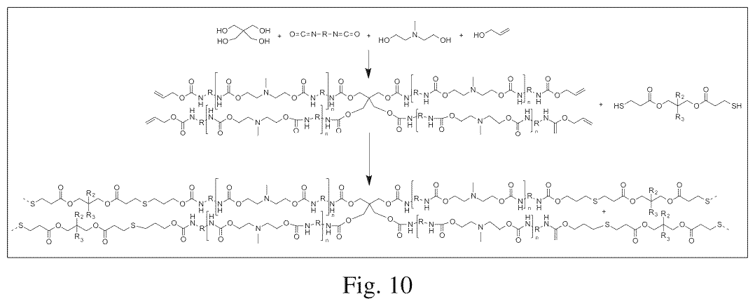

Embodiments include branched oligomers (liquid) plus linear or branched crosslinker(s) in solution that produce a solid thermoset reaction product. See, for example, FIG. 10, which depicts an embodiment of branched tertiary amine macromers with alkene termination reacted with dithiol or polythiol monomers for thermoset polymer fabrication.

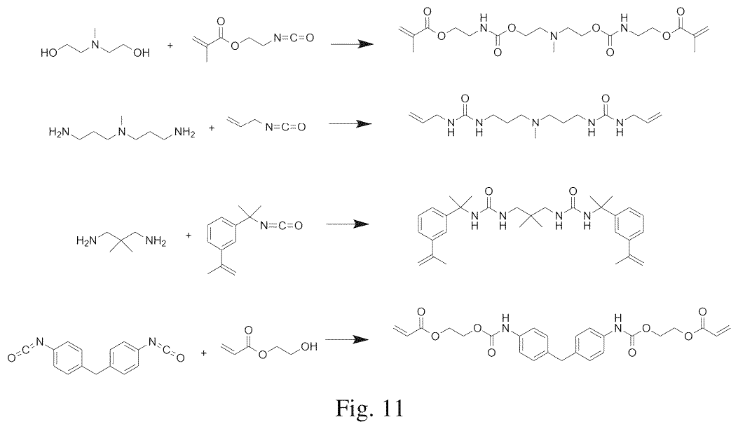

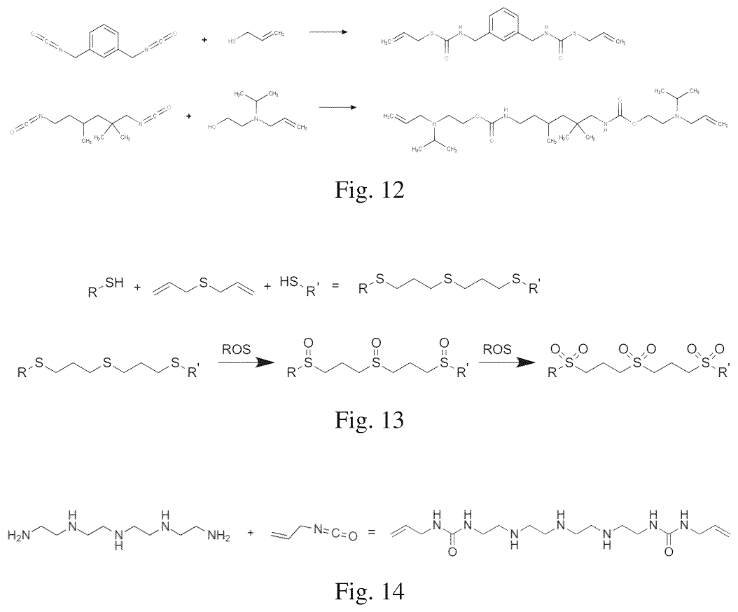

Intermediate macromers from FIGS. 9 and 10 can be replaced with other alkene or alkyne terminated macromers, such as the examples in FIGS. 11 and 12. FIG. 11 depicts embodiments of interchangeable monomer schemes to produce alkene terminated macromers with urethane, urea, aliphatic, and aromatic linkages. These monomers can be used with polythiols to form polymer networks. For example, in UV cured stereolithography resins or device coatings, or redox initiated thiol-ene resins cured in bulk. FIG. 12 depicts embodiments of interchangeable monomer schemes to produce alkene or alkyne terminated macromers with thiourethane, urethane, and tertiary amine linkages. These macromers can be used with polythiols to form polymer networks via thiol-ene click chemistry derived thioether linkages between the reactive monomers.

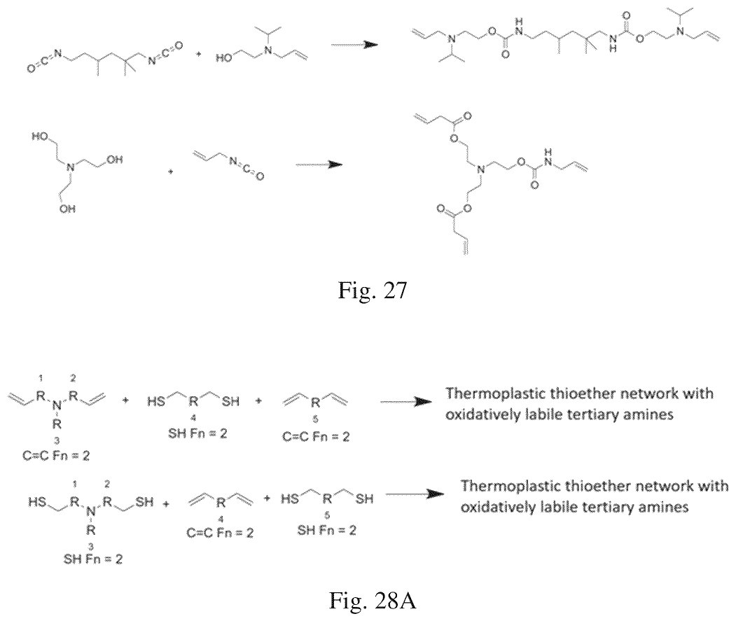

FIG. 27 depicts examples of alkene terminated, tertiary amine containing macromers useful for thermoset and thermoplastic systems.

Examples of tertiary or secondary amine containing alkene crosslinkers include Triallylamine. Tertiary or secondary amine containing chain extenders include diallylmethylamine, and diallylamine. These can be incorporated into thiol-ene polymer systems to impart oxidative degradation in-vivo.

Additional ROS sequestration can be included in a thiolene system, independent of degradation kinetics, by using sulfide containing chain extenders, such as allyl sulfide. These monomers, when incorporated into the polymer network, will sequester reactive oxygen species to form sulfone and sulfoxide functionalities while remaining biodurable. Conversion of sulfides to new oxidative states can be monitored via FTIR wavenumbers. The starting thioether or sulfide containing network would have CH2-S peaks ca. 710-685 cm⁻¹ and/or CH3-S peaks ca. 660-630 cm⁻¹. As the network oxidizes with exposure to ROS (either in vivo, or an in-vitro H₂O₂ solution), sulfoxide peaks would develop ca. 1070-1030 cm⁻¹ and sulfone (S=O) peaks would develop ca. 1350-1300 cm⁻¹, 1160-1120 cm⁻¹.

FIG. 13 depicts an embodiment of synthesis of sulfide macromers for increased ROS sequestering when subsequently polymerized into a polymer system.

In alternative embodiments, the tertiary amine in the backbone of a diamine terminated monomer can be replaced by a secondary amine to still maintain oxidative degradation and ROS scavenging benefits. Example secondary amine containing monomers terminated with end groups that have disparate reaction rates with isocyanates are tetraethylenepentamine, and N-(2-Hydroxytheyl)ethylenediamine. In this embodiment, the increased reactivity of the primary amine end groups can be utilized for alkene end-capping using allyl isocyanate. This alkene terminated monomer is oxidatively labile and can be incorporated into thiolene crosslinked systems (thermoplastic or thermoset) to impart oxidative biodegradation. The alkene terminated monomer would react with thiol terminated monomers. Alkene termination using allyl isocyanate can also be employed for tertiary amine containing diamines and polyamines such as Tris(2-aminoethyl)amine and 3,3′-Diamino-N-methyldipropylamine.

FIG. 14 depicts an embodiment of a secondary amine containing alkene terminated macromer synthesis. Subsequent polymerization yields an oxidatively labile secondary amine containing backbone.

Large molecular weight thermoplastics would be useful for traditional melt processing techniques (extrusion, supercritical gas foam blowing, fused deposition modeling, injection molding, solvent casting, dip coating, spray coating, etc.). Viscous oligomer or macromer solutions could be used for 3D printing, or thermoset resin formulations when combined with additional reactive crosslinkers (epoxies, polyisocyanates, or radical initiated (UV or heat) alkene reactions such as thiol-ene, methacrylate, or acrylate addition; or alkyne addition to primary amines or thiols). Tertiary amines in the macromer backbone could serve as catalysts for urethane or urea forming reactions. This self-catalyzation could be used for the synthesis of thermoplastic resins, reactive injection molding of thermoplastic or thermoset systems, and gas blowing of thermoset polymer foams.

In one embodiment, the dual degradation pathways of oxidative degradation (for example: via tertiary amines in the PU backbone or crosslinkers) and hydrolytic degradation (for example: via ester groups in thiolene-derived crosslinks or ester containing chain extenders) could be leveraged in a biomedical device, such as drug delivery or force transfer. Hydrolytically labile crosslinks could be tuned to degrade more acutely than the oxidative labile linkages to initially decrease modulus as crosslink density decreases. This comparatively rapid change in modulus could enable easier implantation of a high elastic modulus device, but a subsequent drop in modulus could result in a compliance matched implant compared to the implant tissue. Eventually, crosslinks will degrade until a thermoplastic or oligomeric mass is left in situ, at which point the thermoplastic backbone begins to degrade into small terminal degradation products that can be metabolized or cleared from the tissue. The kinetics of each degradation pathway could be tuned to occur in parallel, in series, or with oxidative degradation occurring prior to hydrolytic degradation. These kinetics are tuned with crosslink density, bulk hydrophobicity, pendant group functionalization, and secondary intermolecular forces like hydrogen bonding. For thiolene reacted polymers, hydrolytic scission rate occurs as a function of local sulfide linkage oxidation. In this embodiment, ROS sequestration can occur independent or in conjunction with oxidative degradation when used in the absence or presence of tertiary amine groups, respectively. Sulfide oxidation can also augment hydrolysis of crosslinks or the polymer backbone.

The upper portion of FIG. 15 depicts embodiments of configurations of biomaterials with single and double biodegradation mechanisms. The lower portion of FIG. 15 shows comparative hydrolytic rates based on the degree of material oxidation.

Degradable linkages can be associated with block copolymer macromer sections with varying functional properties (such as hydrophobicity). For example, hydrolytically labile linkages could be incorporated into more hydrophilic repeat units to increase degradation rate and facilitate physical incorporation of soluble hydrophilic therapeutic agents. In the same system, the oxidative degradable motifs could be associated with a more hydrophobic oligomer repeat unit for delayed reaction and physical incorporation of more hydrophobic therapeutic agents that are released approximately at the time of the oxidatively labile repeat unit degradation. In this manner, a multi-stage delivery of therapeutic agents can occur based on degradation rates. As an example, estrogen can be released from a bone graft or gelling depot injection for treating osteoporosis. Estradiol can be incorporated into the polymer network by first forming alkene terminated reactive macromers via esterification with molecules terminated with acyl halides, (meth)acryloyl chloride, or carboxylic acids while also incorporating terminal alkene groups. These reactive estradiol macromers are subsequently reacted with combinations of polythiol crosslinkers, dithiols, polyalkenes, and/or dialkenes to form a polymer network that incorporates estrogen into a medical implant. After polymer implantation, the location of the ester relative to the sulfide linkage and hydrophobicity of the polymer network will determine the rate of ester hydrolysis and liberation of estrogen from the implant. Incorporating a variety of hydrolytically labile therapeutic agent macromers with differing rates of hydrolysis can enable a broader window of therapeutic release over the lifetime of the implant.

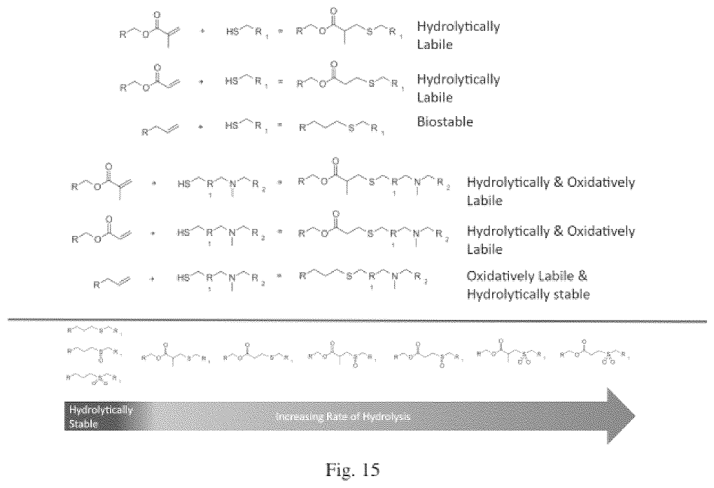

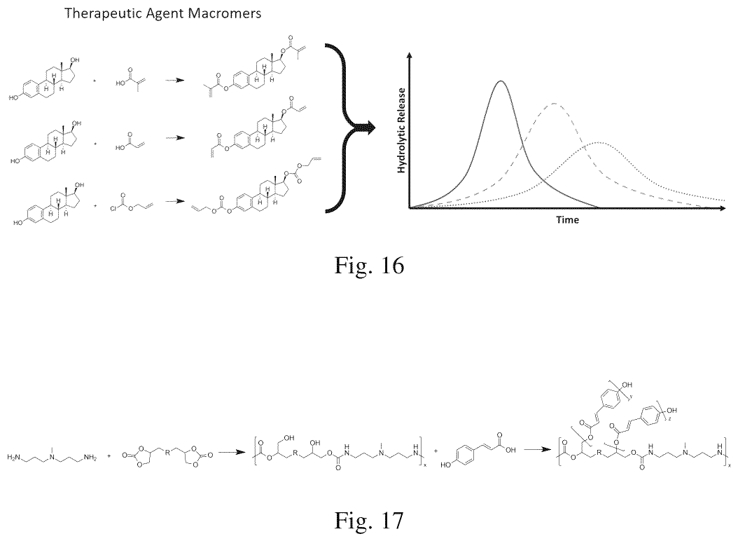

FIG. 16 depicts an embodiment of a population of hydrolytically labile macromers with differing rates of hydrolytic release of a therapeutic agent. By combining all 3 into a material system, the implant has a broader window of therapeutic release.

The inverse could also be employed, where the hydrolytically labile linkages (such as esters) could be incorporated into the hydrophobic segment and oxidative linkages in the hydrophilic segment. In this scenario, the hydrolytic linkages would erode via a surface degradation mechanism(s) because the hydrophobicity of the block segment would prevent water intrusion for bulk hydrolysis.

In an alternative embodiment, the tertiary amine containing monomer (primary amine terminated) could be reacted with a cyclic carbonate monomer or (bis(trichloromethyl) carbonate. Alternatively, cyclic carbonate groups can be incorporated directly into the oligomer backbone via cycloaddition of CO₂ to epoxide terminal groups. The reaction product would be non-isocyanate polyurethanes with oxidatively labile tertiary or secondary amine linkages in the polymer backbone and, in the case of cyclic carbonates, functional pendant groups via primary alcohols. Subsequent pendant functionalization of these alcohols could occur via esterification with carboxylic acid terminated modifiers or urethane linkages with isocyanate terminated modifiers. Additional multifunctional monomers (such as di, tri, or tetra functional amines) can be added to change the functional properties of the oligomers (i.e., solubility parameters, rheology, etc.) or the final thermoset (toughness, hydrophobicity, Tg, modulus etc.) This composition would be especially useful in green chemistry and in materials where surface functionalization is required.

FIG. 17 depicts an embodiment of isocyanate-free synthesis of a tertiary amine containing an oxidatively labile thermoplastic polymer and an example of subsequent functionalization with an antimicrobial agent via carboxylic acid esterification. Other functional groups and molecules could be added to the pendant hydroxyl groups.

In an embodiment, ROS labile polyurethane ureas or polythiourethane ureas may be used to create osteosynthesis implants, soft tissue fixation devices, and bone fillers. The elastic modulus of these materials range from 10 MPa to 30,000 MPa to be comparable with both trabecular and cortical bone (21 MPa-20,000 MPa). In particular, one method to change the elastic modulus is through altering the degree of hydrogen bonding. Increasing bidentate urea linkages for example can lead to an order secondary bonding phase that acts as a rigid filler within the polymer matrix. Conversely, an increase in thiourethane bonds can decrease the elastic modulus as the bond length is longer than that of urethanes and ureas, allowing for more rotation. The use of thiourethane groups also has an advantage in implant systems where hydrophilicity should be avoided. The sulfur atom makes the thiourethane group more hydrophobic, and as a result decreases the likelihood of the polymer matrix being solvated and softening in an aqueous environment. This resistance to moisture plasticization makes this system especially useful for chronic bone fixation implants. This embodiment has the benefit of relatively easy curing and processing conditions when compared to other engineering plastics used in orthopedic applications, such as polysulfone and polyether ether ketone (PEEK). These materials can be cured in-situ in their final shape, synthesized into a thermoplastic and manufactured into a finished part via traditional thermoplastic processing methodologies, cast as a solid thermoset and machined, cast as a thermoset in the final part geometry via reactive injection molding, cast as a porous thermoset using emulsion templating, cast as a porous thermoset using porogen templating, chemically blown into a thermoset foam, or blown into a thermoplastic foam using supercritical gas extrusion (nitrogen, carbon dioxide, etc.). In another thermoset embodiment, the polymer can be formed into a tissue scaffold with macroporosity using porogen templating (sintered wax beads, sintered salt crystals, sintered water-soluble polymer beads, etc.) and microporosity from an emulsion used to cast the macroporous structure.

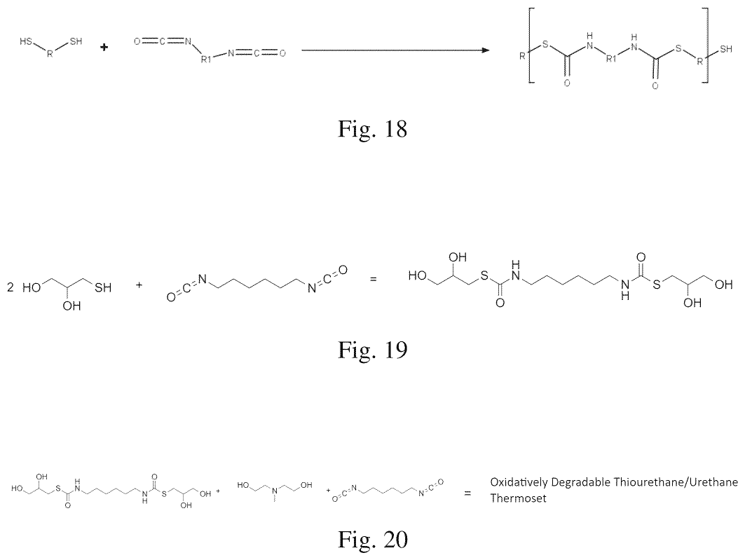

FIG. 18 depicts an embodiment of a thiourethane reaction.

Oxidatively degrading Polythiourethane-urethane systems can also be synthesized in a two-step approach. First, hydroxyl and thiol terminated crosslinkers such as 3-mercapto-1,2-propanediol, 1-thioglycerol, or DL-Dithiothreitol (DTT) are reacted with diisocyanates. The isocyanates may react with the thiols to form hydroxyl-terminated thiourethane monomers/oligomers (depending on the stoichiometric ratios of OH+SH to NCO in the first reaction). Subsequently, these hydroxyl terminated crosslinking macromers are reacted with a tertiary amine containing polyol and a diisocyanate to form an oxidatively degradable thiourethane-co-urethane thermoset network. Alternatively, the hydroxyl terminated thiourethane oligomer can be combined with tertiary amine containing diamines and diisocyanate to form oxidatively degradable thiourethane-co-urea thermoset networks.

FIG. 19 depicts an embodiment of synthesis of thiourethane macromers from thioglycerol and HDI.

FIG. 20 depicts an embodiment of synthesis of an oxidatively labile, tertiary amine containing thermoset using a thiourethane macromer, tertiary amine containing diol, and an aliphatic diisocyanate.

Alternative polythiourethane-urethane systems can be synthesized via a one-pot reaction wherein a thiol-terminated monomer such as glycol dimercaptoacetate, butane dithiol, 3-mercapto-1,2-propanediol, 1-thioglycerol, DL-Dithiothreitol (DTT), pentaerythritol tetrakis(3-mercaptopropionate), trimethylolpropane tris(3-mercaptopropionate), or 2-hydroxymethyl-2-methyl-1,3-propanediol tris-(3-mercaptopropionate) is deprotonated using a tertiary amine monomer such as triethanolamine or N-methyldiethanolamine, or a teriary amine catalyst such as triethylamine to react with isocyanates and yield a high molecular weight thermoplastic or tough thermoset. The resulting polymer networks can yield high-modulus materials that are oxidatively degradable. Embodiments can be cured with or without organic solvents.

For example, the combination of pentaerythritol tetrakis(3-mercaptopropionate), trimethylhexamethylene diisocyanate (TMHDI), and triethanolamine yields a thermoset network with an elastic modulus of 1.5-2.5 GPa at 37 C. In an alternative embodiment, a two-step reaction can be employed. First, the thiol terminated monomers can be reacted with excess isocyanate monomers in the presence of an alcohol terminated tertiary amine catalyst/monomer to yield a viscous prepolymer or quasi-prepolymer. Second, the balance of the reactive equivalent monomers is added to the prepolymer. Reactive monomers can be hydroxyl, thiol, amine, or carboxylic acid terminated for network formation. The reactive monomers can contain tertiary amines to catalyze the second reaction and enable oxidative degradation in the final polymer network. Water can also be added for chemical blowing. This method can be employed to create solid polymeric parts, emulsion templated constructs, or gas blown foam components comprised of an oxidatively labile polythiourethane-co-urea/urethane polymer.

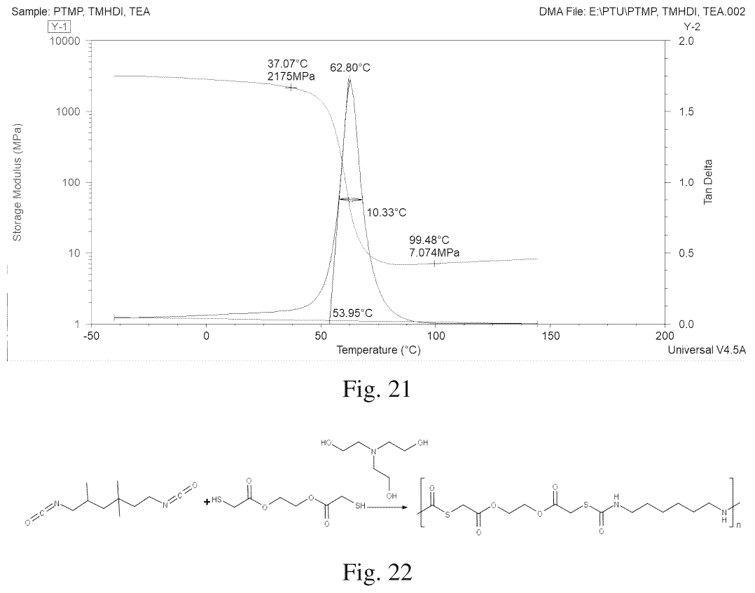

FIG. 21 depicts an embodiment of a DMA curve of a thermoset polythiourethane polymer.

In another example, an aliphatic linear polythiol urethane can be synthesized via a one pot reaction using a dithiol monomer (such as glycol dimercaptoacetate) and an aliphatic diisocyanate (such as TMHDI) at a reactant ratio of 1:0.7 to 1:1.2, ideally 1:1.01 (thiol:isocyanate equivalents) in an organic solvent (such as isopropanol or dimethylsulfoxide) at a volume ratio of 1:2 to 1:20, ideally 1:10 reactants to solvent. Components are mixed using an overhead stirring unit between 300-1000 rpm, and in an embodiment, about 600 rpm, until well mixed, 1-15 minutes. 10-1000 ppm of a base catalyst, such as triethanolamine or N-methyldiethanolamine, is added to the vessel under continuous stirring. The resulting reaction proceeds until the polythiourethane precipitates out of the reaction solvent. The remaining solvent is then decanted and the polymer is placed in a vacuum oven at −32 inHg at 80 C for 48 hours. The resulting polymer is a tough thermoplastic polythiourethane with a Tg between 20-45 C. The polymer can be further processed through melt spinning, casting, or dissolved in solvents such as THF, DMF, DMAc, or DMSO.

In an alternative embodiment, the dithiol and diisocyanate monomers are reacted in an organic solvent that does not react with isocyanate or thiol functional groups (such as tetrahydrofuran) and a monofunctional chain terminator to reduce molecular weight (such as allyl alcohol or allyl isocyanate) to prevent precipitation. The alkene terminated, thiourethane repeat unit prepolymer can be subsequently reacted with other prepolymer macromers to create a block copolymer.

FIG. 22 depicts an embodiment of synthesis of a hydrolytically responsive linear aliphatic polythiourethane repeat unit derived from diisocyanate and dithiol monomers catalyzed with a tertiary amine group.

### Bone Graft Embodiments:

UV curable, tertiary amine containing urethane, urea, or thiourethane macromers are synthesized from diols, diamines, dithiols with 2-isocyanatoethyl methacrylate, allyl isocyanate, or 3-Isopropenyl-α,α-dimethylbenzyl isocyanate. Example dithiol monomers include ethylene glycol bis-mercaptoacetate, 2,2′-(Ethylenedioxy)diethanethiol, 2,2′-Thiodiethanethiol, tetra(ethylene glycol) dithiol, 1,2-ethanedithiol, 1,3-propanedithiol, 1,4-butanedithiol, 1,6-butanedithiol, 1,8-Octanedithiol, 1,11-Undecanethiol. Aromatic macromers can be synthesized for hardening the bone graft system. Macromer synthesis can be via diol or diamine synthesis with 3-Isopropenyl-α,α-dimethylbenzyl isocyanate or, alternatively, aromatic diisocyanate synthesis with an alkene terminated monofunctional alcohol, amine, carboxylic acid, or thiol. Alkene terminated macromers with tertiary amine functionality can be synthesized by reacting an isocyanate monomer (such as trimethylhexamethylene diisocyanate) with 2-[Allyl(isopropyl)amino]ethanol, as shown in FIG. 12. Other curable thiol-ene embodiments can include off the shelf polyfunctional alkene terminated monomers including 1,4-Butanediol divinyl ether, 1,4-Cyclohexanedimethanol divinyl ether, 3,9-Divinyl-2,4,8,10-tetraoxaspiro[5.5]undecane, 1,2,4-Trivinylcyclohexane, Allyl ether, Diallylamine, Diallyl carbonate, Tri(ethylene glycol) divinyl ether, Trimethylolpropane diallyl ether, Di(ethylene glycol) divinyl ether, 1,9-Decadiene, 1,5-Hexadiene, 1,3-Butadiene, 1,4-Pentadiene, 1,4-Pentadien-3-ol, 2-Methyl-1,3-butadiene, 2,3-Dimethyl-1,3-butadiene, 1,7-Octadiene, and isoprene. The index of refraction for the continuous phase and external phases can be engineered to serve as an internal light guide (using internal refraction of the curing UV light) to propagate light through all features of complex emulsion features. This manufacturing process would enable the incorporation of porous HIPE features as overmolded components on a piece of orthopedic hardware and integrating with through-holes, blind ledges, slots, T-slots, and other mechanical interface features. Alternatively, radical initiation could occur through redox initiators mixed into the HIPE resin at the time of manufacturing for full volume cure. After implantation, osteoconduction through the porous polymer scaffold and subsequent biodegradation of the scaffold would result in neobone formation around, through, and with the orthopedic hardware, resulting in stronger fixation and integration with the orthopedic appliance.

For any proposed polymerization system (condensation or radical polymerization) with any proposed functional groups (urethane, urea, thiourethane, amide, ester, ether, tertiary amine, sulfide, acrylate, methacrylate) and any degradation site (tertiary amine, ester, carbonate, ether), the molecular weight between degradation sites can range between 76 daltons for the shortest hyrolytically degrading repeat unit up to 50 kDa for tertiary amine degrading systems. This 50 kDa molecular weight between crosslinks is the upper limit for glomerulus filtration of degradation products from systemic circulation. Other embodiments can exceed a molecular weight of 50 kDa between degradation sites where the implant application and target tissue facilitate other forms of degradation product clearance such as metabolization and endocytosis of degraded material particulates. Degradation sites can be along a thermoplastic backbone, at covalent crosslink sites in a thermoset, or along polymer chains connecting covalent crosslinks in a thermoset system. Degradation products can be linear or branched oligomers. For all oxidatively degradable embodiments, the material will undergo complete gravimetric mass loss when exposed to an oxidative environment, such as immersion in a hydrogen peroxide solution. Embodiments with sulfide linkages may undergo initial mass gain due to incorporation of oxygen into the network, but hydrolytically or oxidatively labile linkages also present in the network would ultimately contribute to full gravimetric mass loss over time. Embodiments incorporating tertiary amine degradadable linkages shall include FTIR signatures for the C—N stretch between 1250-1020 cm-1 for aliphatic tertiary amines and 1335-1250 cm-1 for aromatic amines. If the polymer is exposed to an acid (such as an in-vitro hydrochloric acid solution), the polymer could form a tertiary amine salt with FTIR peaks ca. 2700-2300 cm⁻¹ with an absence of bending peaks from 1625-1500 cm⁻¹.

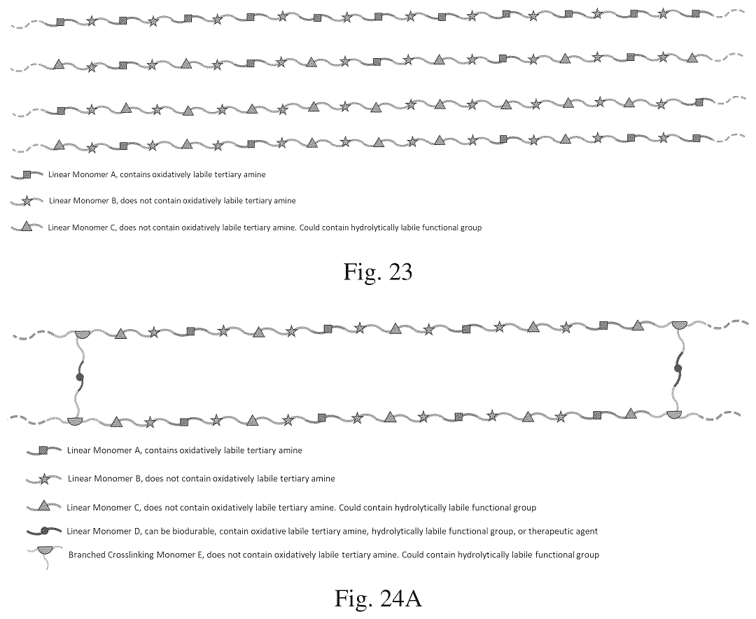

FIGS. 23-25 graphically depict generic polymer networks derived from/including embodiments. The synthetic schemes described herein depict an oxidatively degradable material platform that enables selective control of the polymer architecture. Specifically, control over the location of degradable tertiary amine linkages, for controlled degradation rate and terminal degradation products intended for bioresorption and ultimate clearance from the implant tissue site and excreted from the body. Dotted lines indicate connections to the greater polymer network. Repeat units length and molecular weights between crosslinks are arbitrary and could span molecular weights previously described. These are illustrative networks and do not include an exhaustive list of monomer combinations. FIG. 23 depicts an embodiment of polymer networks with short degradable repeat unit thermoplastics, medium degradable thermoplastic repeat units from copolymers, or long degradable thermoplastic repeat units.

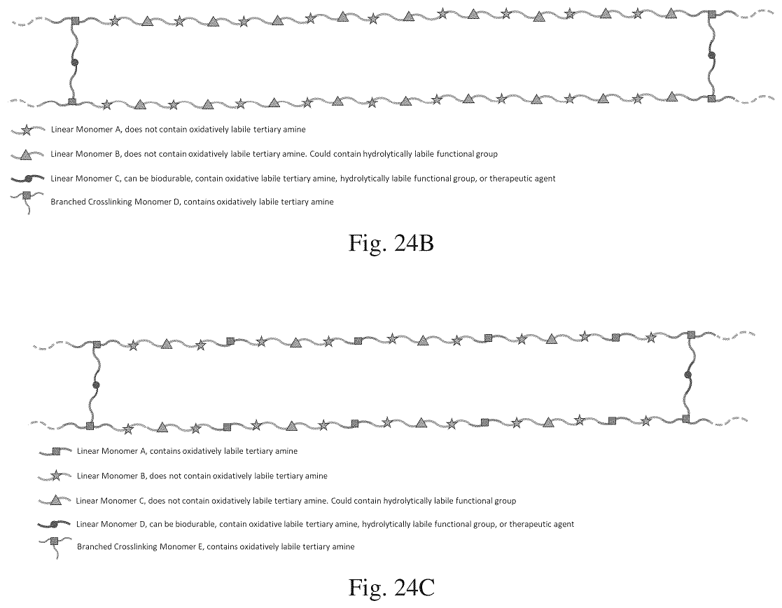

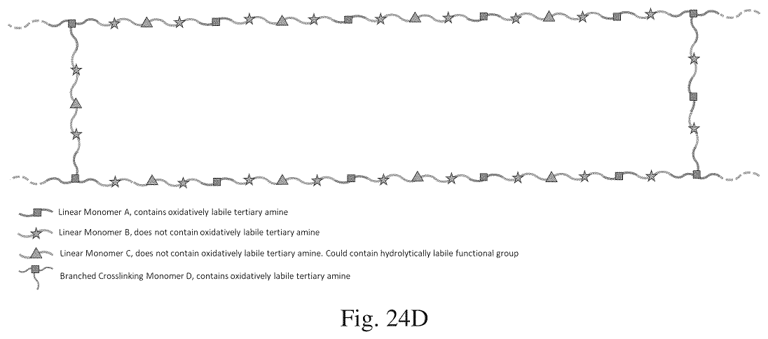

FIGS. 24A, 24B, 24C, 24D depict embodiments of polymer architectures that include thermoplastic chains that have been subsequently crosslinked or lightly crosslinked thermosets to form elastomeric materials.

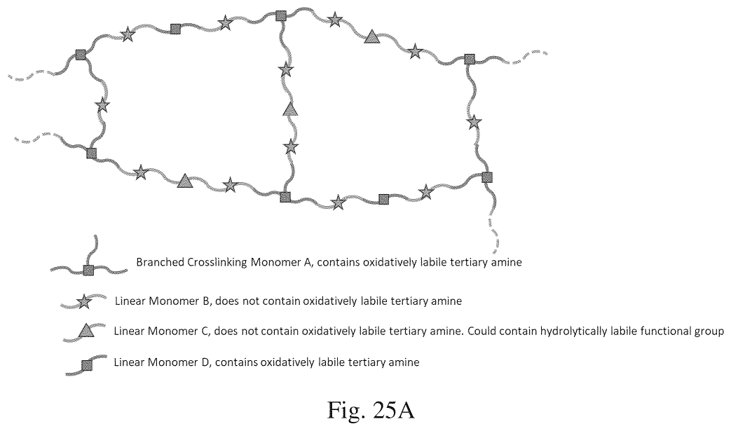

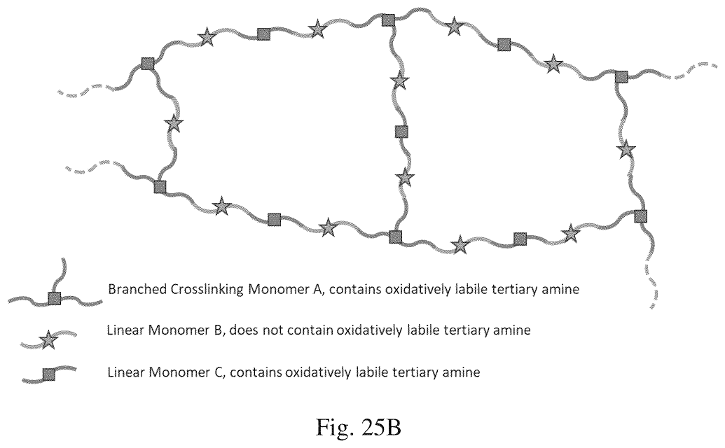

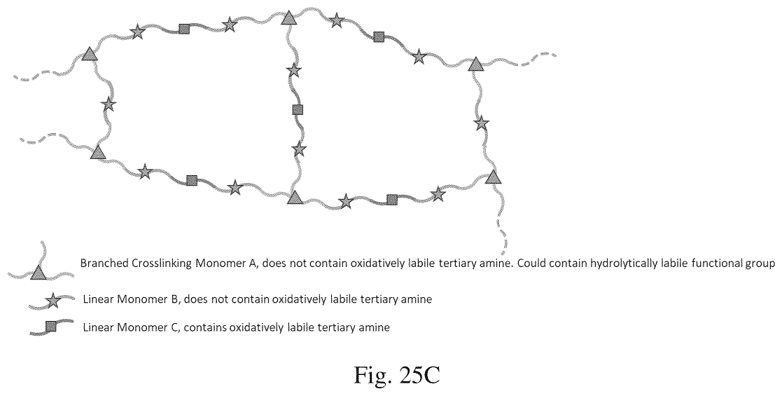

FIGS. 25A, 25B, 25C depict embodiments that include highly crosslinked thermosets.

Brief Examples of Device Embodiments Using the Described ROS Scavenging Material Systems:
1. Thermoplastic Injection molded or reactive injection molding polymer orthopedic anchors for ligament fixation. High modulus anchor with softer over-molded features for ligament/tendon insertion sites into bone. Alternatively, high modulus anchor with recessed features to accommodate soft tissue tendon/ligament interface.
2. Extruded biodurable hydrocephalus shunts or glaucoma shunts with sulfide linkages for ROS scavenging.
3. Polymer surgical sutures or meshes. Non-hydrolytic degradation mechanism provides benefit with mitigating acidic degradation products. High modulus suture serves as a staple analog. Meshes can be woven or crossing filaments are melted together where they overlap.
4. Traditional implant coatings to promote tissue integration and beneficial healing response by scavenging ROS. Examples include polymer coatings for interfacing implants like dialysis portals and pedicle screws, a porous annulus for percutaneous access site sealing, and orthopedic plate or screw coatings.
5. Polymer drug delivery vehicle, such as a cancer drug depot injection in high ROS tumor environment.
6. Polymer gastric plug. Polyureas with tertiary amines in the backbone could provide toughness in the acidic gastric environment. Separate repeat units or co-extruded polymers could differentially oxidize in the gastric environment to facilitate force transfer to native tissue for durable sealing.
7. Injectable gel for rheumatoid arthritis and other ROS generating diseases that impact synovial joints.
8. Injectable gel and/or microparticles for localized and site-specific treatment/management of osteopenia and osteoporosis of the spine, femoral neck, and other key susceptible sites.
9. Foam bone scaffold implant in trabeculectomy.
10. Load bearing, compressible thermoplastic elastomers. 3D printed via fused deposition modeling, direct ink write, or stereolithography. Alternatively, injection molded as solid material or porous via supercritical gas or reactive gas foaming. Useful as tissue fillers.
11. Porous polymer tissue scaffolds via supercritical gas extrusion with Nitrogen or carbon dioxide.
12. Polymeric vascular access seals. Elastomeric foams, sutures filaments, or elastomeric solids.
13. Porous polymeric Lung seals for procedures that compromise the lung pleura.
14. Polymer Hemostatic or traumatic wound packing dressings.
15. Polymer tenodesis anchor that expands when deployed through reversible or irreversible methods
   a. Example: Delivery device heats anchor above Tg for delivery in rubbery regime to conform to anatomy. Implant modulus increases when implant cools to body temperature.
16. Thermoplastic or thermoset polymer Interference Screw.
17. Thermoplastic or thermoset polymer Craniofacial osteosynthesis plates and screws.
18. Polymer Joint arthrodesis implant.
19. Thermoplastic or thermoset fracture and arthrodesis fixation devices.
20. Sheaths for vessel anastomosis—blood, nerve, lymph.
21. Surgical sutures. ROS sequestering and or degradable thermoplastic fibers. Urethane/Urea and or Urethane/Thio-urethane block copolymers for increased toughness.
22. Surgical meshes. Woven thermoplastic fibers or thermoset foam meshes. Can be used as hernia meshes or screw interface sleeves with bone.

### Detailed Examples of Device Embodiments Using the Described ROS Scavenging Material Systems:

Example 1a—Diabetic wound dressing: N-methyldiethanolamine is mixed with a diisocyanate (e.g., hexamethylene diisocyanate) and other aliphatic diols in a polar solvent (e.g., THE or Methylene chloride) to form a high molecular weight thermoplastic. The thermoplastic polymer solution is electrospun into a fiber mat, cleaned, dried, packaged, and radiation sterilized as a diabetic ulcer wound dressing. Once implanted into the diabetic ulcer site, the woven mesh integrates into the tissue. Localized ROS from the wound site react with and are neutralized by the tertiary amine in the N-methyldiethanolamine derived portion of the thermoplastic repeat unit. The polymer degrades in response to the local ROS in the interstitial fluid of the wound, in addition to the ROS generated by immune cells responding to the foreign implant. The implant is fully degraded and replaced by native tissue.

Example 1 b—The wound dressing described in 1a with additional ROS scavenging capability by incorporating sulfide linkages. This polymer formulation could include the reaction product of trimethylhexamethylene diisocyanate, N-butyldiethanolamine, 3,6-Dithia-1,8-octanediol and other diols and/or diamines for structure property relationships. Sulfide and tertiary amine content can be blended to independently control the ROS sequestration capacity, degradation rate, and size of degradation oligomers depending on the target therapeutic application.

Example 2a—Long time-scale degrading ligament fixation: An aliphatic block copolymer is synthesized in an anhydrous polar solvent by combining an aliphatic diisocyanate (Hexamethylene diisocyanate or trimethylhexamethylene diisocyanate) with diamines (i.e. 3,3′-Diamino-N-methyldipropylamine for the degradable tertiary amine, and other diamines for structure property relationships) to form the polyurea hard segment, and diols (i.e. N-butyldiethanolamine for the degradable tertiary amine, and other diols for structure property relationships) to form the polyurethane soft segment. The polymerized thermoplastic polymer is extruded under tension to create strain hardened, axially aligned filaments with increased tensile toughness for ligament fixation applications. Hydrophobic groups can be incorporated into the polymer backbone to minimize moisture plasticization and maintain hydrogen bonding to mitigate creep. The oxidative degradation mechanism allows for matched degradation and tissue back-filling for effective force transfer in this load bearing device application.

Example 2b—In an alternative ligament fixation embodiment of 2a, the linear thermoplastic repeat unit includes thiourethane linkages for increased toughness. In this embodiment, linear diols and/or diamines are replaced with dithiol monomers such as 2,2′-Thiodiethanethiol or 2,2′-(Ethylenedioxy)diethanethiol.

Example 2c—In an alternative ligament fixation device embodiment of 2a or 2b, after fiber formation, unsaturated carbon groups in the thermoplastic polymer backbone or thermoplastic pendant groups (derived from trimethylol propyl allyl ether, and/or N-Allyl-2,2′-Iminodiethanol included in the thermoplastic synthesis) can be radiation crosslinked to provide covalent crosslinks to mitigate creep. This material crosslinking can occur in the context of final device sterilization. Sterilization and crosslinking can be done with Gamma, Ebeam, or Xray sterilization modalities. Parts can be processed in a cold chain to limit thermal accumulation and keep parts below glass and melting transition temperatures during sterilization.

Example 3a—Biodegradable vascular stents: A biodegradable vascular stent that degrades via tertiary amine linkages in the polymer network. The polymer can be thermoplastic, thermoset, or processed as a thermoplastic and subsequently crosslinked into a thermoset with radiation. The polymer is formed in a tubular shape, laser machined into the stent geometry, and radially crimped/pleated/folded onto a delivery system. The delivery system provides radial expansion and/or thermal energy to heat the polymer above the glass transition temperature to expand the device while in the rubbery regime of the elastic modulus. The ideal implant has a moisture plasticized glass transition above 37 C or a hydrophobic polymer network that resists moisture plasticization in-vivo and has a glass transition temperature above 37 C. A device with a transition temperature above 37 C is implanted in the polymer's “glassy plateau” and highest elastic modulus state. This maximizes radial force and hoop compression for the stent and reduces the minimum wall thickness required to keep the vessel patent. Reducing the wall thickness reduces the cross-sectional area of the device impeding blood flow. The advantage of the oxidatively degrading system over other more traditional hydrolytic systems is the dovetailed progression of tissue healing and material mass loss.

Example 3b—During manufacturing of the device in 3a, the thermoset polymer stent is heated above the glass transition temperature during radial crimping onto the delivery system and cooled below the glass transition temperature in this compressed configuration. Shape setting the polymer onto the delivery system reduces the stress required from the delivery system (either via balloon inflation or other means) to expand the device in the glassy regime of the elastic modulus when delivering below the glass transition temperature. The thermoset polymer tube that is machined into the stent geometry can be formed via thermoplastic extrusion or electrospinning a tubular structure and subsequent radiation crosslinking; reactive injection molding and/or machining; or dip coating of a reactive resin or UV curable resin.

Example 3c—In an alternative embodiment of a vascular stent, the machined extruded polymer tube is replaced by small diameter, tertiary amine containing, oxidatively degrading, elastic polymer filaments (0.002-0.02″ OD) that are woven into a mesh tube device geometry. In an ideal embodiment, the polymer elastic modulus is optimized to match the compliance of the extruded fiber diameter to the target vascular tissue compliance. Small diameter fiber cross sections could benefit from the high elastic modulus of a block polyurea-co-urethane thermoplastic that is the reaction product of hexamethylene diisocyanate, 1,4-Bis(3-aminopropyl)piperazine, and other diols and diamines for optimized structure property relationships. The device can be delivered endovascularly as a stent or flow diverting device and the increase in device diameter is dependent on elastic recovery of the radially compressed filaments.

Example 3d—In an alternative embodiment of 3c, the high elastic modulus block co-polymer thermoplastic fibers are replaced by high elastic modulus thermoset fibers. These fibers could be extruded as a viscous solution in a solvent and evaporatively drawn as a thermoplastic (linear polymer reacted from isophorone diisocyanate, N-tert-butyl diethanolamine, 5-Norbornene-2,2-dimethanol and other difunctional diols and/or diamines for structure property relationships. Filaments can be heat crosslinked during the drawing process, or radiation crosslinked.

Example 4a—Orthopedic anchors: A porous 3D printed anchor for use in tenodesis, securing sutures, or suture tape. The 3D printable resin is comprised of UV curable macromers with alkene and thiol function groups where urethane, urea, or thiourethane groups may exist in the macromer backbone such as diurethane dimethacrylate. Other macromers or monomers include 1,4-butanedithiol or pentaerythritol tetrakis(3-mercaptopropionate). Using vat, clip, or other methods of 3D printing, the resulting polymer is a thermoset polyurethane/thiourethane. To enhance tissue integration, the monolith will include micro/nano surface features such as pores or surface roughness. Additionally, the anchor includes macro features like threads or barbs that help secure into the bone and prevent loosening or migration. As time progresses, the material will degrade in response to the local ROS response, eventually yielding a healing response comprised of neotissue formation in place of the degraded material. The incorporation of osteoconductive moieties such as hydroxyapatite can be employed to enhance bone formation by mixing into the resin at weight percents ranging from 5-40%.

Example 4b—In an alternative embodiment of 4a, the UV curable 3D printing resin contains a branched macromer (urea, urethane, or thiourethane containing) terminated with >2 alkene groups or >2 thiol groups. The resin also contains other polythiol terminated and polyalkene terminated monomers. In this embodiment, all monomer selections avoid the incorporation of ester groups in the final, UV cured polymer network (i.e., methacrylate, acrylate, or mercaptopropionate derived esters) and intentionally include a tertiary amines in the polymer network for oxidative degradation of the biomaterial. Example tertiary amine containing macromers include the reaction product of a diisocyanate (such as TMHDI) and 2-[Allyl(isopropyl)amino]ethanol or the reaction product of Tris(2-aminoethyl)amine with allyl isocyanate. Alkene terminated macromers are reacted with polythiol monomers (SH functionality 2) via UV curing in the presence of a UV initiator or via Redox initiator systems.

Example 5—Osteoporosis/Osteopenia Femoral Neck Stabilizer: Intramuscular injections to surround the femoral neck and sequester ROS contributing to the loss of bone density. Thermal gelation at body temp stabilizes the material to remain in the local injection area. The injection can include estrogen for sustained release to locally help reverse the progression of osteopenia without the effects of systemic estrogen replacement therapy.

Example 5b—Hydrophilic polysulfide-co-ether thermoplastic oligomers are synthesized from combinations of allyl ether, 2,2′-Thiodiethanethiol, Tetra(ethylene glycol) dithiol, and diethylene glycol monoallyl ether (DGME) for chain termination. Oligomer synthesis is conducted with slight excess of dithiol monomers and DGME concentration is varied to control macromer molecular weight. The oligomer solution is purified through a column to yield oligomers terminated with one hydroxyl and one thiol with a monodisperse molecular weight. A triblock copolymer is synthesized via thiol-ene addition of the thiol terminated hydrophilic oligomers to a hydrophobic core segment of poly(propylene glycol)diacrylate. The triblock copolymer is used for a thermal gelation depot injection for sequestering local tissue ROS.

Example 5c—Hydrophobic polythioester oligomers are synthesized via thiol-ene addition of 2,2′-Thiodiethanethiol and isoprene in solution and the chain terminator allyl alcohol in solution with an organic solvent. The oligomer solution is purified through a column to yield a monodisperse population of alcohol terminated hydrophobic polysulfide oligomers in organic solution. The hydroxyl groups are functionalized with 2-Isocyanatoethyl Acrylate. The dilute organic solution of hydrophobic polythioester oligomers is starve-fed with Acrylate-PEG-NHS in a Michael addition between the acrylate groups. Alternatively, the acrylate groups are combined under UV radiation in the presence of a UV initiator or an alternative radical initiation scheme. The resulting triblock copolymer has a hydrophobic, methylated polysulfide core with hydrophilic PEG flanking chains.

Example 5d—A triblock polymer composed of hydrophilic and hydrophobic oligomers can be synthesized through condensation reactions. For example, PEG dithiol varying in molecular weight from ˜200 Da to 50 kDa can be dissolved in a solvent such as THE or toluene. Using stannous octoate or other catalyst to create a reactive thiolate to which propylene sulfide can be added to for ring opening polymerization. The final product yielded is a triblock polymer with a PEG based core and polypropylene sulfide ends. Alternatively, a step growth method using PEG-dithiol and polypropylene sulfide (PPS) in an addition reaction where the stoichiometric ratio between the PEG dithiol and PPS is 1:2.1 or greater, and in an embodiment, about 1:10. In a reaction vessel with PPS, dissolved in solvent or performed in bulk, a catalyst and PEG can be added dropwise while vigorously mixing. The resulting polymer can be separated from the reactants through solvent exchange, filtration, extraction, thermal precipitation, or evaporation yielding a triblock polymer.

Example 6a—Orthopedic staple: High toughness, minimal creep, thermoset thiourethane or thiourethane-co-urethane. Incorporation of urethane and thiourethane linkages for a bone staple impart toughness in the polymer network based on the secondary interactions (hydrogen bonding) that allow the material to undergo large strain values while maintaining desired mechanical properties. Additionally, an added benefit of the thermoset system is the resistance to creep while exerting compressive loads exceeding 20N to achieve active compression during healing. The final staple device is then loaded onto a delivery device by straining the device from a primary to secondary shape within the materials elastic region, packaged and terminally sterilized using either standard ionizing radiation techniques, EtO or alternative gases. Final kit configurations include at minimum the implant and delivery device in a sterilized configuration and ready for implantation. For implantation, the device is inserted into premade holes that accept the implant. After the implant is fully seated, it will return to the primary configuration and actively compress the bone segments together even in the event of bone resorption or poor initial reduction.

Example 6b—A thermoset polythiourethane polymerized via casting in bulk using a branched, thiol terminated monomer such as pentaerythritol tetrakis(3-mercaptopropionate) is deprotonated using a tertiary amine such as N-methyldiethanolamine or triethanolamine to react with a diisocyanate. The functionality of the thiol monomer(s) and tertiary amine moiety can be modulated to achieve the desired mechanical properties via crosslink density or physical crosslinks. The monomer mixture can be reactive injected molded into a mold and cured under vacuum at <50 C for 48 h to yield a polythiourethane based bone staple that sequesters ROS at the thioester linkage and hydrolytically degrades at the mercaptopropionate derived ester group.

Example 6c—A thermoset polythiourethane-co-urethane synthesized in a two-step reaction. First, thioglycerol is combined with a diisocyanate at a 1:1 NCO:SH ratio in the presence of a tertiary amine catalyst (such as N-methyldiethanolamine) to create a tetrafunctional polyol thiourethane macromer. This macromer is subsequently added to a diisocyanate and a tertiary amine containing diol (such as N-methyldiethanolamine) at a 1-4% NCO equivalent excess to yield a thermoset material. The reactive resin can be reactive injection molded into a staple geometry, or cured in a bulk pre-form and subsequently machined into a staple geometry.

Example 7—The embodiments of 6a-c used for a spinal fusion cage.

Example 8—Wound Dressing: A hydrophilic aliphatic polyurethane is polymerized in an anhydrous polar solvent using hexamethylene diisocyanate, a tertiary amine containing diol such as N-methyldiethanolamine, and other diols for structure-property relationships (such as polyether diols for increased hydrophilicity) and other difunctional monomers such as functionalized therapeutic macromers or diols with unsaturated carbon pendant groups for subsequent ebeam sterilization. The thermoplastic polymer is purified and cast into an implant-grade film free of any manufacturing aids such as catalysts or surfactants typically found in HIPE's (High Internal Phase Emulsion) or gas blown foam porous structures. The film may include biocompatible, high surface energy particulate fillers to serve as physical blowing cell nucleating agents. The film is saturated with supercritical gasses such as nitrogen or carbon dioxide. Rapidly depressurizing the film physically blows the film into an implant-grade closed cell foam. The foam is packaged and irradiated with ebeam, gamma, or x-ray radiation to both sterilized the material and covalently crosslink the material to impart elastic resilience in the foam. Once resilient, the part is compressed in the sterile packaging to disrupt the cell membranes and create an open celled structure that can be used as a biodegradable, exudate absorbing wound dressing with or without antimicrobial properties.

Example 9a—Bone Scaffolding: Porous polyHIPE scaffolds can be fabricated using an emulsion-templating technique where insoluble solutions are mixed to create a biphasic dispersion. The dispersions are often oil-in-water, water-in-oil, and oil-in-air. To prevent unwanted swelling in certain applications, a water-in-oil emulsion may be used. In this example, the oil (organic phase) represents the continuous phase containing precursor molecules, surfactants and if needed diluents such as a non-reactive solvent. The organic phase can be comprised of reactive monomers or prepolymers at various ratios. For example, the combination of acryloyl groups such as 1,4-butanediol acrylate (0.010-0.100 mmol, in an embodiment, about 0.044 mmol), urethane acrylates, such as diurethane diacrylate (0-.005-.1 mmol 0.011 mmol) or a diurethane macromer derived from the reaction of n-methyldiethanolamine and 2-Isocyanatoethyl Acrylate, and if desired a thiol terminated moiety such as pentaerythritol tetrakis(3-mercaptopropionate) (0.009-0.100 mmol, and in an embodiment, about 0.018 mmol). Addition emulsion stabilizing elements including a dilutant such as toluene (10-80 wt %, and in an embodiment, about 50%), and or surfactant, such as PGPR 4125 (1-20 wt %, and in an embodiment, about 5%). Finally, an initiator can be incorporated into the continuous phase. The initiator can be any initiator that propagates radical initiation. This can be through temperature, reduction-oxidation, or electromagnetic radiation. UV initiators such as BAPO at concentrations of (ranging from 0.00025 to 0.01 mmol, and in an embodiment, about 0.001 mmol) may be used as they can allow for rapid curing overcoming the obstacles of a semi-stable emulsion. Prior to internal phase incorporation, the constituents of the continuous phase should be well mixed. For example, substances can be added to a 100 mL polypropylene mixing cup and mixed for 1-60 minutes at 500 rpm with an overhead stirring unit. Once well mixed, the mixing speed can be adjusted based on the desired pore size, and in an embodiment, less than 100 rpm. Incorporation of the internal phase is achieved dropwise in a controlled manner until the volume fraction of the aqueous to organic phase exceeds 74.04%, however can range from 74.4%-99% and in an embodiment, about 80-90% with pore sizes ranging from 10 microns to 647 microns. A dilute salt solution like 1 wt % CaCl2 is used to prevent Oswald ripening. The resulting high internal emulsion (HIPE) can be mixed for an additional time to maintain the droplet formation and coalescence prior to polymerization. Polymerization can be performed and monitored using FTIR by assessing peaks at 840, 935, 1635 cm-1 or a combination thereof to confirm conversion of reactive methacrylates. For example, in a UV cured system, the HIPEs were cured for 15 minutes at a wavelength of 365 nm, removed from the mixing cup, inverted, and cured for an additional 15 minutes to yield fully polymerized system with degree of conversion exceeding 90%. After polymerization, the excess aqueous phase and toluene can be mechanically removed from the porous polyHIPEs and washed with solvents such as isopropanol and placed under vacuum for at least 48 hours at elevated temperature such as 80° C. The washed polyHIPE material is packaged and sterilized for shipment. The sheet of sterile porous material is a bone scaffold that can be cut and shaped at the time of implantation to optimize fit to the patient anatomy and desired therapeutic application (bone fusion, hardware integration, etc.).

Example 9b—The emulsion system of example 9a poured over a sintered porogen template (moisture sintered salt crystals, wax beads, water soluble beads) and cured. After removing the porogens with water or wax, the resulting tissue scaffold has microporosity from the porogens, and microporosity within the struts from the HIPE resin.

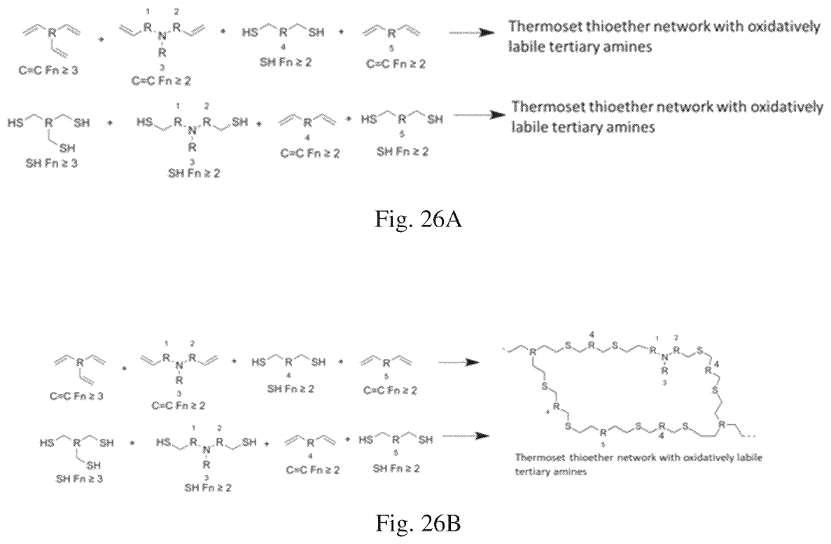

Example 9c—The emulsion system of example 9a where the macromer system is replaced with a branched polyalkene (such as triallyl amine), a linear dithiol (such as 1-4 butanethiol) and a reactive macromer (such as 3,3′-diamino-N-methyldipropylamine terminated with allyl isocyanate to create a dialkene macromer with a tertiary amine group). The polymer network in this embodiment would consciously omit any ester linkages, and biodegradation of the polymer network would occur exclusively due oxidation of tertiary amines in the polymer architecture. Refer to FIG. 26 where all R groups do not include any ester linkages. R groups can include urethane, thiourethane, and/or urea linkages. The stoichiometry could be biased to include excess alkene or thiol groups for subsequent network functionalization. Residual thiols would be FTIR detectable with SH peaks ca. 2600-2550 cm⁻¹. FIG. 26A is an embodiment of a generic implementation of an emulsion/HIPE system, with adding a tertiary amine to make it oxidatively degradable. An embodiment includes esters in the R group (mercaptopropionates leading to oxidative and hydrolytic degradation) of the polythiol monomer. Another embodiment has no esters in the R group of any monomer (including the polythiol) to make the final cured polymer entirely oxidatively degradable and hydrolytically stable. FIG. 26A shows two possible routes to arrive at a thermoset thioether network with oxidatively labile tertiary amines. FIG. 26B depicts an example of the scheme of FIG. 26A. FIG. 26B shows a thermoset thioether network with oxidatively labile tertiary amines that may be generated via either path of FIG. 26B.

Example 10—Fabrication of an interpenetrating network comprised of two independent polymer networks. In one example, the first networks is a ROS degradable network formed by a condensation reaction between N-methyldiethanolamine, 2,2,4-Treimtheyl-1,6-hexamethylene diisocyanate and triethanolamine. The second network is solution blended by means of thermal or solvent. The second network is comprised of macromers that react via a thiol-click reaction such as pentaerythritol tetrakis(3-mercaptopropionate) and a urethane methacrylate that can be independently polymerized using radical, reduction-oxidation, or Michael addition. The resulting polymer system allows for combinatory networks with two distinct properties; such as combining high elongation with a high elastic modulus.

The foregoing description of the embodiments of the invention has been presented for the purposes of illustration and description. It is not intended to be exhaustive or to limit the invention to the precise forms disclosed. This description and the claims following include terms, such as left, right, top, bottom, over, under, upper, lower, first, second, etc. that are used for descriptive purposes only and are not to be construed as limiting. For example, terms designating relative vertical position refer to a situation where a side of a substrate is the “top” surface of that substrate; the substrate may actually be in any orientation so that a “top” side of a substrate may be lower than the “bottom” side in a standard terrestrial frame of reference and still fall within the meaning of the term “top.” The term “on” as used herein (including in the claims) does not indicate that a first layer “on” a second layer is directly on and in immediate contact with the second layer unless such is specifically stated; there may be a third layer or other structure between the first layer and the second layer on the first layer. The embodiments of a device or article described herein can be manufactured, used, or shipped in a number of positions and orientations. Persons skilled in the relevant art can appreciate that many modifications and variations are possible in light of the above teaching. Persons skilled in the art will recognize various equivalent combinations and substitutions for various components shown in the Figures. It is therefore intended that the scope of the invention be limited not by this detailed description, but rather by the claims appended hereto.

What is claimed is:

1. A composition comprising:
a thermoplastic polymer comprising repeat units;
wherein each repeat unit includes (a) a tertiary amine, and (b) at least one of a urethane linkage, a thioether linkage, a urea linkage, or combinations thereof.

2. The composition of claim 1, wherein each repeat unit includes a urethane linkage.

3. The composition of claim 1, wherein each repeat unit includes a thioether linkage.

4. The composition of claim 1, wherein each repeat unit includes a urea linkage.

5. The composition of claim 1, wherein each repeat unit includes a urethane linkage and thioether linkage.

6. The composition of claim 1, wherein each repeat unit includes a urethane linkage and a urea linkage.

7. The composition according to claim 2, wherein the thermoplastic polymer includes a hydrolytically labile functional group.

8. The composition according to claim 2, wherein:
the thermoplastic polymer includes a polymer backbone;
the polymer backbone includes the repeat units.

9. The composition according to claim 2, wherein:
each repeat unit includes a pendent group;
the pendent group includes an alkene.

10. The composition of claim 9, wherein the pendent group is pendent from the tertiary amine.

11. The composition of claim 9, the polymer comprising polymerized monomers, the monomers including at least one of 2,2′-[1,2-Ethanediylbis(2-propenylimino)]bisethanol; 2-[Allyl(2-hydroxyethyl)amnio]ethan-1-ol; N-Allyl-2,2′-Iminodiethanol; or combinations thereof.

12. A composition comprising:
a crosslinked thermoset polymer comprising a tertiary amine and repeat units;
wherein each repeat unit includes at least one of a urethane linkage, a thioether linkage, a urea linkage, a thioether linkage, or combinations thereof;
wherein the polymer includes a branched crosslinking element.

13. The composition of claim 12, wherein the branched crosslinking element includes the tertiary amine.

14. The composition of claim 12, wherein one of the repeat units includes the tertiary amine.

15. The composition of claim 14, wherein the branched crosslinking element includes another tertiary amine.

16. The composition of claim 12, wherein the branched crosslinking element includes a hydrolytically labile functional group.

17. An apparatus comprising:
a porous fiber mat;
wherein the fiber mat includes a polymer that comprises repeat units;
wherein each repeat unit includes a tertiary amine and a urethane linkage;
wherein the polymer is oxidatively degradable.

18. The apparatus of claim 17, wherein the polymer is a crosslinked thermoset polymer.

19. The apparatus of claim 17, wherein the polymer is a thermoplastic.

20. The apparatus according to claim 17, wherein the fiber mat is an electrospun scaffold.

21. The apparatus according to claim 17, wherein the polymer includes no ester linkages.

22. The apparatus according to claim 17, wherein the polymer includes an ester linkage.
